(19) United States
(12) Patent Application Publication
(10) Pub. No.: US 2024/0350708 A1
(43) Pub. Date: Oct. 24, 2024

(54) POROUS BONE SUBSTITUTION MATERIAL

(71) Applicants: UNIV PARIS XIII PARIS-NORD VILLETANEUSE, VILLETANEUSE (FR); UNIVERSITE PARIS CITE, PARIS (FR); OST DEVELOPPEMENT, CLERMONT-FERRAND CEDEX 2 (FR); CENTRE NATIONAL DE LA RECHERCHE SCIENTIFIQUE, Paris (FR); ETAT FRANCAIS REPRESENTE PAR LA DIRECTION CENTRALE DU SERVICE DE SANTE DES ARMÉES, Paris (FR)

(72) Inventors: Didier LUTOMSKI, MONTIGNY LE BRETONNEUX (FR); Geraldine ROHMAN, DEUIL LA BARRE (FR); Sylvie CHANGOTADE, OTHIS (FR); Jean-Marc COLLOMBET, BRETIGNY SUR ORGE (FR); Sophie FRASCA, BREUILLET (FR); Hubert GUIBOURT, VOLVIC (FR); Credson LANGUEH, CHATILLON (FR); Anne CONSALUS, DRANCY (FR)

(21) Appl. No.: 17/997,439

(22) PCT Filed: May 20, 2021
(86) PCT No.: PCT/EP2021/063493

§ 371 Date: Jun. 9, 2023

(30) Foreign Application Priority Data
May 20, 2020 (FR) 2005385

(51) Int. Cl. A61L 27/36; A61F 2/28; A61F 2/30; A61L 27/18; A61L 27/44; A61L 27/56

(52) U.S. Cl. CPC A61L 27/3608; A61F 2/3094; A61L 27/18; A61L 27/44; A61L 27/56; A61F 2002/2835; A61F 2002/30784

(57) ABSTRACT

The present invention relates to a porous bone substitution material for bone repair, in particular the repair of a cavitary bone defect and/or the repair of a segmental bone defect.

### TECHNICAL FIELD

The present invention belongs to the field of bone substitute materials intended for bone repair, in particular repair of a cavitary bone defect and/or repair of a segmental bone defect.

### PRIOR ART

Bone is continuously subjected to a renewal and repair process. Indeed, our bone capital thus adapts to the biomechanical stresses of our existence, replacing old tissue with new tissue. Bones are composed of cells, osteocytes, surrounded by a mineralized extracellular matrix. This matrix is renewed thanks to the equilibrium between the action of two types of cells: osteoblasts and osteoclasts. Osteoblasts synthesize the bone matrix, while osteoclasts remove the aging bone tissues under the effect of various hormones and mechanical stresses. This process gives the bone surprising self-repairing properties, making it capable of regenerating in the event of injury. Thus, after a fracture, the realignment and maintenance of the limb is generally sufficient for healing: by generating new tissue, the osteogenesis process fills in the deficit due to the fracture, restoring the functional efficacy of the bone.

However, in some cases, this natural self-healing process is insufficient: about once out of ten, mechanical or biological problems prevent fracture self-healing. Moreover, certain bone lesions encountered in victims of domestic accidents or road accidents, of attacks, certain pathologies (such as pseudo-arthrosis) or surgical interventions (ablation of tumours, cysts, infected sites) may result in significant losses of bone substance that natural osteogenesis will not suffice to fill in. Bone must then be assisted.

One contemplated solution for repairing bone is to graft an autologous bone fraction. We then speak of bone autograft. The autograft does not produce a defence immune response since the tissue comes from the patient. However, it results in considerable cell death in the transplanted tissue. The ability of the graft to produce new bone cells can compensate for this loss, but it depends in particular on the vascularization of the graft. The latter is in fact essential to bone in reconstruction: the vessels provide the energy and the nutrients necessary for cell proliferation. Furthermore, autografting requires two surgical sites (harvesting then grafting) which can cause complications (pain, abscesses, neuralgia). The size of the graft required for filling in represents another important limit.

Another envisaged solution for bone reparation is to graft a bone fraction originating from a donor.

These two solutions are not satisfactory. That is why it appears necessary to develop a bone substitute material having properties analogous to natural bone, but also capable of promoting osteosynthesis associated with growth factors, progenitor cells.

To allow the repair of a bone defect, the bone substitute material must have two important properties:
osteoconduction, which is the capacity of the material to serve as a passive support for bone regrowth, and
osteoinduction, property of a material containing proteins, the release of which induces the biological cascade necessary for bone formation.

It must further be porous and resorbable.

Several types of materials are used as passive support for cell and tissue colonization: natural or synthetic ceramics, or various materials of natural origin, the chemical composition of which approximates that of the mineral phase of the bone, may be used. Materials of natural origin originate from various sources: by way of example, on can cite ceramicized bovine bone or coral exoskeleton (porites), a calcium carbonate which has interesting osteoconductive and biomechanical properties. The most often used synthetic materials for filling in bone defects are hydroxyapatite and tricalcium phosphates, two mineral species of the phosphate family, pure or as a mixture. They may be prepared in the form of blocks or granules. Controlling density, grain size, and porosity will determine the behaviour of the material in vivo.

However, when they are used, these materials cannot be used alone and need to be associated with growth factors, progenitor cells. In addition, these materials do not allow a restoration of the continuity and of the bone structure in large bone defects and rarely combine the osteoconduction and osteoinduction properties with a controlled and complete biodegradability, in harmony with the bone regeneration kinetics.

Document US2011/268782 describes an osteoimplant comprising non-decellularized bone particles, in particular bone particles of bovine origin and a thermosetting, non-elastomeric and non-porous polyurethane resin.

The inventors have therefore developed a new porous bone substitute material having good osteoconduction and good osteoinduction while having good biocompatibility and degradability adapted to bone regeneration. In addition, the porous bone substitute material can easily be manipulated by the surgeon and can easily be shaped to adapt to all types of bone defects, including important bone defects.

### SUMMARY OF THE INVENTION

Thus, a subject of the present invention is therefore a porous bone substitute material comprising:
at least one porous elastomer matrix, and
particles of decellularized bone.

Another subject of the present invention is the use of said porous bone substitute material in bone repair, preferably the repair of a bone cavity defect and/or the repair of a segmental bone defect.

Another subject of the present invention is a bone repair kit comprising the porous bone substitute material.

Another subject of the present invention is a method for preparing a bone substitute material.

### DETAILED DESCRIPTION OF THE INVENTION

The present invention thus relates to a porous bone substitute material comprising:
at least one porous elastomer matrix, and
particles of decellularized bone.

Within the meaning of the present invention, the term “bone substitute material” means a physical support on which osteoprogenitor cells can adhere, migrate, proliferate and differentiate into osteoblasts, which are cells responsible for bone formation, at the surface and inside the bone substitute material.

Advantageously, the bone substitute material according to the invention is a composite material comprising at least one porous elastomer matrix and particles of decellularized bone, which individual properties combine to form a heterogeneous material (the bone substitute material) having highly improved overall performance, properties which are not observable with the at least one elastomer matrix or decellularized bone particles, when used individually.

The inventors have surprisingly shown that the porous bone substitute material comprising at least one elastomer matrix and decellularized bone particles according to the invention has:
mechanical properties sufficient to withstand the force stresses, but also the regeneration process in the area to be repaired and to be a support for the bone tissue in this area,
a porosity and an interconnectivity allowing the circulation of progenitor cells, nutrients and other molecules involved in the regularizations of these processes, while allowing internal vascularization of the porous bone substitute material of the invention,
a roughness allowing cell adhesion and adsorption of molecules involved in the regularizations of these processes.

More particularly, the inventors have shown that the porous bone substitute material is both mechanically stable (Young's modulus E of 100-300 kPa), non-toxic (metabolic activity of mesenchymal stromal cells greater than 90% after 24 hours of incubation with the porous bone substitute material according to the invention), biodegradable (lifetime at 37° C. comprised between 12 and 65 months) and osteoconductive. Indeed, the inventors have shown that the porous bone substitute material allows the adhesion of progenitor cells as well as their proliferation, including in depth, followed by the differentiation of progenitor cells into osteoblasts.

Within the meaning of the present invention, the term “elastomer matrix” is intended to mean a structure consisting of a porous elastomer system, said structure being capable of including the decellularized bone particles. Advantageously, the at least one elastomer matrix according to the present invention has good biodegradability, good biocompatibility and good mechanical properties.

Advantageously, the isocyanate index of the elastomer matrix is between 0.1 and 6.0. Advantageously, the isocyanate index is between 0.1 and 5.0, advantageously between 0.2 and 4.9, advantageously between 0.3 and 4.8, advantageously between 0.4 and 4.7, advantageously between 0.5 and 4.7, advantageously between 0.6 and 4.6, advantageously between 0.7 and 4.5, advantageously between 0.8 and 4.5, advantageously between 0.9 and 4.5, advantageously, between 1 and 4.5, advantageously between 1.05 and 4.5, advantageously between 1.1 and 4.5, advantageously between 1.2 and 4.5, advantageously between 1.3 and 4.5, advantageously between 1.4 and 4.5, advantageously between 1.5 and 4.5, advantageously between 2.0 and 4.5, advantageously between 2.5 and 4.5, advantageously between 2.6 and 4.4, advantageously between 2.7 and 4.3, advantageously between 2.8 and 4.2, advantageously between 2.9 and 4.1, advantageously between 3.0 and 4.0.

Within the meaning of the present invention, the term “elastomer” is intended to mean one or more crosslinked polymers having “rubber elasticity” properties. In a particular embodiment of the invention, the elastomer must be biocompatible and biodegradable. Advantageously, the Young's modulus in compression of the bone substitute material of the invention is between 10 kPa and 1000 kPa.

Within the meaning of the present invention, “biocompatible” elastomer matrix means an elastomer matrix which is advantageously both compatible for an implantation in a patient, that is to say that this implantation has a benefit/risk ratio that is favourable from the therapeutic point of view, for example within the meaning of Directive 2001/83/EC, that is to say a risk that is reduced or even non-existent for the patient, versus the concerned therapeutic benefit; and compatible for including therein decellularized bone particles, that is to say that it allows the inclusion of the decellularized bone particles, that it does not or only slightly degrade the activity of the decellularized bone particles included in the matrix, and that is suitable for bone reconstruction once the bone substitute material is implanted in a patient, human or animal.

Within the meaning of the present invention, the term “biodegradable” elastomer matrix is intended to mean an elastomer matrix which is bioresorbable and/or biodegradable and/or bioabsorbable, with a common goal of progressive disappearance, with one or more different or complementary mechanisms of degradation, solubilization or absorption of the elastomer matrix in the patient, human or animal, in which the material has been implanted.

In a particular embodiment of the invention, the at least one elastomer matrix according to the invention comprises an elastomer based on poly(ester-urea-urethane).

In a particularly advantageous embodiment of the invention, the at least one elastomer matrix of the porous bone substitute material according to the invention comprises an elastomer based on poly(ester-urea-urethane), the ester being chosen from caprolactone (PCL) oligomers, lactic acid (PLA) oligomers, glycolic acid (PGA) oligomers, hydroxy butyrate (PHB) oligomers, hydroxyvalerate (PVB) oligomers, dioxanone (PDO) oligomers, Poly(ethylene adipate) (PEA) oligomers, poly(butylene adipate) oligomers (PBA) or combinations thereof.

In a particular embodiment, the at least one elastomer matrix of the porous bone substitute material is a matrix comprising a poly(caprolactone-urea-urethane)-based elastomer. In another particular embodiment, the at least one elastomer matrix of the porous bone substitute material is a matrix comprising a poly(lactic acid-urea-urethane)-based elastomer. In another particular embodiment, the at least one elastomer matrix of the porous bone substitute material is a matrix comprising a poly(glycolic acid-urea-urethane)-based elastomer. In another particular embodiment, the at least one elastomer matrix of the porous bone substitute material is a matrix comprising a poly(hydroxyvalerate-urea-urethane)-based elastomer. In another particular embodiment, the at least one elastomer matrix of the porous bone substitute material is a matrix comprising a poly(hydroxybutyrate-urea-urethane)-based elastomer. In another particular embodiment, the at least one elastomer matrix of the porous bone substitute material is a matrix comprising a poly(dioxanone-urea-urethane)-based elastomer. In another particular embodiment, the at least one elastomer matrix of the porous bone substitute material is a matrix comprising a poly(ethylene adipate urea urethane)-based elastomer. In another particular embodiment, the at least one elastomer matrix of the porous bone substitute material is a matrix comprising a poly(butylene adipate urea urethane)-based elastomer.

In a particular embodiment, the at least one elastomer matrix of the porous bone substitute material is a matrix comprising an elastomer based on poly(caprolactone-urea-urethane) and poly(lactic acid-urea-urethane). In a particular embodiment, the at least one elastomer matrix of the porous bone substitute material is a matrix comprising an elastomer based on poly(caprolactone-urea-urethane) and poly(glycolic acid-urea-urethane). In a particular embodiment, the at least one elastomer matrix of the porous bone substitute material is a matrix comprising an elastomer based on poly(caprolactone-urea-urethane) and poly(hydroxyvalerate-urea-urethane). In a particular embodiment, the at least one elastomer matrix of the porous bone substitute material is a matrix comprising an elastomer based on poly(caprolactone-urea-urethane) and poly(hydroxybutyrate-urea-urethane). In a particular embodiment, the at least one elastomer matrix of the porous bone substitute material is a matrix comprising an elastomer based on poly(caprolactone-urea-urethane) and poly(dioxanone-urea-urethane).

In one embodiment, the at least one elastomer matrix of the porous bone substitute material is a matrix comprising an elastomer based on poly(caprolactone-urea-urethane) and poly(ethylene adipate-urea-urethane). In a particular embodiment, the at least one elastomer matrix of the porous bone substitute material is a matrix comprising an elastomer based on poly(caprolactone-urea-urethane) and poly(butylene adipate-urea-urethane).

In a particular embodiment, the at least one elastomer matrix of the porous bone substitute material is a matrix comprising an elastomer based on poly(lactic acid-urea-urethane) and poly(glycolic acid-urea-urethane). In a particular embodiment, the at least one elastomer matrix of the porous bone substitute material is a matrix comprising an elastomer based on poly(lactic-urea-urethane acid) and poly(hydroxyvalerate-urea-urethane). In a particular embodiment, the at least one elastomer matrix of the porous bone substitute material is a matrix comprising an elastomer based on poly(lactic-urea-urethane acid) and poly(hydroxybutyrate-urea-urethane). In a particular embodiment, the at least one elastomer matrix of the porous bone substitute material is a matrix comprising an elastomer based on poly(lactic-urea-urethane acid) and poly(dioxanone-urea-urethane).

In a particular embodiment, the at least one elastomer matrix of the porous bone substitute material is a matrix comprising an elastomer based on poly(lactic-urea-urethane acid) and poly(ethylene adipate-urea-urethane). In a particular embodiment, the at least one elastomer matrix of the porous bone substitute material is a matrix comprising an elastomer based on poly(lactic-urea-urethane acid) and poly(butylene adipate-urea-urethane).

In a particular embodiment, the at least one elastomer matrix of the porous bone substitute material is a matrix comprising an elastomer based on poly(glycolic acid-urea-urethane) and poly(hydroxyvalerate-urea-urethane). In a particular embodiment, the at least one elastomer matrix of the porous bone substitute material is a matrix comprising an elastomer based on poly(glycolic acid-urea-urethane) and poly(hydroxybutyrate-urea-urethane). In a particular embodiment, the at least one elastomer matrix of the porous bone substitute material is a matrix comprising an elastomer based on poly(glycolic acid-urea-urethane) and poly(dioxanone-urea-urethane). In a particular embodiment, the at least one elastomer matrix of the porous bone substitute material is a matrix comprising an elastomer based on poly(glycolic acid-urea-urethane) and poly(ethylene adipate-urea-urethane). In a particular embodiment, the at least one elastomer matrix is a matrix. The porous bone substitute material is a matrix comprising an elastomer based on poly(glycolic acid urea urethane) and poly(butylene adipate-urea-urethane).

In a particular embodiment, the at least one elastomer matrix of the porous bone substitute material is a matrix comprising an elastomer based on poly(hydroxyvalerate-urea-urethane) and poly(hydroxybutyrate-urea-urethane). In a particular embodiment, the at least one elastomer matrix of the porous bone substitute material is a matrix comprising an elastomer based on poly(hydroxyvalerate urea urethane) and poly(dioxanone urea urethane). In a particular embodiment, the at least one elastomer matrix of the porous bone substitute material is a matrix comprising an elastomer based on poly(hydroxyvalerate-urea-urethane) and poly(ethylene adipate-urea-urethane). In a particular embodiment, the at least one elastomer matrix of the porous bone substitute material is a matrix comprising an elastomer based on poly(hydroxyvalerate-urea-urethane) and poly(butylene adipate-urea-urethane).

In a particular embodiment, the at least one elastomer matrix of the porous bone substitute material is a matrix comprising an elastomer based on poly(dioxanone-urea-urethane) and poly(ethylene adipate-urea-urethane). In a particular embodiment, the at least one elastomer matrix of the porous bone substitute material is a matrix comprising an elastomer based on poly(dioxanone-urea-urethane) and poly(butylene adipate-urea-urethane).

In a particular embodiment, the at least one elastomer matrix of the porous bone substitute material is a matrix comprising an elastomer based on poly(ethylene adipate-urea-urethane) and poly(butylene adipate-urea-urethane).

In a particular embodiment, the at least one elastomer matrix of the porous bone substitute material is a matrix comprising an elastomer based on poly(caprolactone-urea-urethane), poly(lactic acid-urea-urethane) and poly(glycolic acid-urea-urethane).

In a particular embodiment, the at least one elastomer matrix of the porous bone substitute material is a matrix comprising an elastomer based on poly(caprolactone-urea-urethane), poly(lactic acid-urea-urethane), poly(glycolic acid-urea-urethane) and poly(hydroxyvalerate-urea-urethane).

In a particular embodiment, the at least one elastomer matrix of the porous bone substitute material is a matrix comprising an elastomer based on poly(caprolactone-urea-urethane), poly(lactic acid-urea-urethane), poly(glycolic acid-urea-urethane), and poly(hydroxybutyrate-urea-urethane).

In a particular embodiment, the at least one elastomer matrix of the porous bone substitute material is a matrix comprising an elastomer based on poly(caprolactone-urea-urethane), poly(lactic acid-urea-urethane), poly(glycolic acid-urea-urethane), poly(hydroxyvalerate-urea-urethane) and poly(hydroxybutyrate-urea-urethane). In a particular embodiment, the at least one elastomer matrix of the porous bone substitute material is a matrix comprising an elastomer based on poly(caprolactone-urea-urethane), poly(lactic acid-urea-urethane), poly(glycolic acid-urea-urethane), poly(hydroxyvalerate-urea-urethane), poly(hydroxybutyrate-urea-urethane) and poly(dioxanone-urea-urethane).

In a particular embodiment, the at least one elastomer matrix of the porous bone substitute material is a matrix comprising an elastomer based on poly(caprolactone-urea-urethane), poly(lactic-urea-urethane), poly(glycolic-urea-urethane), poly(hydroxyvalerate-urea-urethane), poly(hydroxybutyrate-urea-urethane), poly(dioxanone-urea-urethane) and poly(butylene adipate-urea-urethane).

In a particular embodiment, the at least one elastomer matrix of the porous bone substitute material is a matrix comprising an elastomer based on poly(caprolactone-urea-urethane), poly(lactic acid-urea-urethane), poly(glycolic acid-urea-urethane), poly(hydroxyvalerate-urea-urethane), poly(hydroxy butyrate-urea-urethane), poly(dioxanone-urea-urethane), poly(butylene adipate-urea-urethane) and poly(ethylene adipate-urea-urethane).

These elastomers indeed allow the implementation of the present invention and furthermore have the advantages of being cytocompatible, of allowing the restoration of the physiological stresses of the deficient bone, of avoiding a reoperation after restoration and of allowing a correct reconstruction of the deficient bone. Particularly advantageously, the at least one elastomer matrix of the porous bone substitute material is a matrix comprising an elastomer based on poly(caprolactone-urea-urethane). This matrix comprising an elastomer based on poly(caprolactone-urea-urethane) also has the advantage of having an elastomer nature, providing the matrix with flexibility and having an interconnected porous structure and osteo-inducing properties suitable for bone reconstruction.

In one embodiment according to the invention, the bone particles present in the porous bone substitute material are decellularized bone particles. Within the meaning of the present invention, the term “decellularized bone” means the bone collagenic matrix consisting exclusively of collagen, in particular of type I collagen, the mineral phase consisting of hydroxyapatite crystals (crystallized calcium phosphate) and calcium carbonate, and osteoinductive proteins. The presence of collagen and osteoinductive proteins allows an increase in osteoconduction and osteoinduction. Advantageously, the decellularized bone is obtained from a spongious natural bone. Advantageously, the spongious natural bone may be a human femoral head.

In other words, the decellularized bone according to the invention is free of bone cells (osteoblasts, osteoclasts, osteocytes and bone flanking cells) and of any potentially pathogenic and/or immunogenic constituents. In a particular embodiment, the proportion of collagen present in a decellularized bone particle is between 10 and 40% by weight, advantageously between 15% and 35%. Advantageously, the decellularized bone particle consists of type I collagen and type III collagen.

Advantageously, a decellularized bone particle comprises, relative to the total weight of the particle:
a proportion of lipids less than 2% by weight,
a proportion of proteins of between 25 and 45% by weight,
a proportion of calcium of 10 to 30% by weight,
a proportion of phosphorus of 5 to 20% by weight,
a water content of less than 15% by weight,
   the calcium/phosphorus ratio advantageously being from 1 to 2.2.

In a particular embodiment, the decellularized bone particles of the porous bone substitute material may be obtained from natural bone. Advantageously, the natural bone particles may be obtained from allogeneic or xenogeneic bone, of human or animal origin.

Advantageously, the decellularized bone particles of the porous bone substitute material may be obtained from natural bone according to one of the methods described in patents FR2798294 or EP0502055.

In a particular embodiment, the decellularized bone particles of the porous bone substitute material according to the invention are obtained from a natural bone of human or animal origin. Advantageously, the decellularized bone particles of the decellularized material are obtained from a spongious natural bone. Advantageously, the spongious natural bone may be a human femoral head.

In a particular embodiment, the decellularized bone particles according to the invention have a diameter of between 1 nm and 1 mm. Advantageously, the diameter of the decellularized bone particles is between 1 nm and 1 mm, advantageously between 10 nm and 900 μm, advantageously between 100 nm and 800 μm, advantageously between 100 nm and 700 μm, advantageously between 100 nm and 600 μm, advantageously between 100 nm and 500 μm, advantageously between 1 μm and 800 μm advantageously between 1 μm and 700 μm, advantageously between 10 μm and 600 μm, advantageously between 100 μm and 550 μm, advantageously between 200 μm and 500 μm, advantageously between 300 μm and 450 μm, advantageously between 300 μm and 400 μm. Advantageously, the diameter of the decellularized bone particles is between 300 μm and 400 μm.

By way of example of decellularized bone, mention may in particular be made of Allodyn® and Osteopure® (OST Development, Clermont Ferrand) for the bone of human origin, or the product Laddec®, (OST Development, Clermont-Ferrand) for the bone of animal origin.

In an advantageous embodiment of the invention, the porous bone substitute material according to the invention comprises:
at least one elastomer matrix comprising an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof, and
particles of decellularized bone.

In a first particular embodiment of the invention, the porous bone substitute material according to the invention comprises:
at least one elastomer matrix comprising an elastomer based on poly(caprolactone-urea-urethane), and
particles of decellularized bone.

In a second particular embodiment of the invention, the porous bone substitute material according to the invention comprises:
at least one elastomer matrix comprising an elastomer based on poly(lactic acid-urea-urethane), and
particles of decellularized bone.

In a third particular embodiment of the invention, the porous bone substitute material according to the invention comprises:
at least one elastomer matrix comprising an elastomer based on poly(glycolic acid-urea-urethane), and
particles of decellularized bone.

In a fourth particular embodiment of the invention, the porous bone substitute material according to the invention comprises:
at least one elastomer matrix comprising an elastomer based on poly(caprolactone-urea-urethane) and on poly(lactic acid-urea-urethane), and
particles of decellularized bone.

In a fifth particular embodiment of the invention, the porous bone substitute material according to the invention comprises:
at least one elastomer matrix comprising an elastomer based on poly(caprolactone-urea-urethane) and on poly(glycolic acid-urea-urethane), and
particles of decellularized bone.

In a sixth particular embodiment of the invention, the porous bone substitute material according to the invention comprises:
at least one elastomer matrix comprising an elastomer based on poly(lactic acid-urea-urethane) and on poly(glycolic acid-urea-urethane), and
particles of decellularized bone.

In a seventh particular embodiment of the invention, the porous bone substitute material according to the invention comprises:
at least one elastomer matrix comprising an elastomer based on poly(caprolactone-urea-urethane), poly(lactic acid-urea-urethane) and poly(glycolic acid-urea-urethane) and
particles of decellularized bone.

In an eighth particular embodiment of the invention, the porous bone substitute material according to the invention comprises:
at least one elastomer matrix comprising an elastomer based on poly(hydroxyvalerate-urea-urethane), and
particles of decellularized bone.

In a ninth particular embodiment of the invention, the porous bone substitute material according to the invention comprises:
at least one elastomer matrix comprising an elastomer based on poly(hydroxy butyrate-urea-urethane), and
particles of decellularized bone.

In a tenth particular embodiment of the invention, the porous bone substitute material according to the invention comprises:
at least one elastomer matrix comprising an elastomer based on poly(dioxanone-urea-urethane), and
particles of decellularized bone.

In an eleventh particular embodiment of the invention, the porous bone substitute material according to the invention comprises:
at least one elastomer matrix comprising an elastomer based on poly(ethylene adipate-urea-urethane), and
particles of decellularized bone.

In a twelfth particular embodiment of the invention, the porous bone substitute material according to the invention comprises:
at least one elastomer matrix comprising an elastomer based on poly(butylene adipate-urea-urethane), and
particles of decellularized bone.

In an advantageous embodiment of the invention, the porous bone substitute material according to the invention consists solely of:
at least one elastomer matrix comprising an elastomer based on poly(ester-urea-urethane), the ester being chosen from caprolactone (PCL) oligomers, lactic acid (PLA) oligomers, glycolic acid (PGA) oligomers, hydroxy butyrate (PHB), hydroxyvalerate (PVB) oligomers, dioxanone (PDO) oligomers, poly(ethylene adipate) (PEA) oligomers, poly(butylene adipate) oligomers (PBA) or combinations thereof, and
particles of decellularized bone.

In an advantageous embodiment of the invention, the porous bone substitute material according to the invention consists of:
at least one elastomer matrix comprising an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof, and
particles of decellularized bone.

Whatever the embodiment mentioned above, the particles of decellularized bone may be obtained from a natural bone of human or animal origin or from a synthetic bone.

Advantageously, the decellularized bone particles having a diameter comprised between 1 nm and 1 mm, advantageously comprised between 300 μm and 400 μm.

In a particularly advantageous embodiment of the invention, the porous bone substitute material according to the invention comprises:
at least one elastomer matrix comprising an elastomer based on poly(caprolactone-urea-urethane), and
particles of decellularized bone.

Advantageously, the inventors have demonstrated that the specific combination of particles of decellularized bone and of at least one elastomer matrix comprising an elastomer based on poly(caprolactone-urea-urethane), confers on the porous bone substitute material a better biocompatibility of the material due to the presence of the hydroxyapatite. Indeed, the degradation of the at least one elastomer matrix comprising an elastomer based on poly(caprolactone-urea-urethane) produces a slightly acidic environment, leading to a decrease in cell proliferation. The addition of the decellularized bone particles makes it possible to neutralize this acidity, thanks to the presence of hydroxyapatite.

The inventors have also brought to light that the specific combination of particles of decellularized bone and of at least one elastomer matrix comprising an elastomer based on poly(caprolactone-urea-urethane) allows an increase in osteoinduction compared to the use of the elastomer matrix comprising a poly(caprolactone-urea-urethane)-based elastomer alone. Indeed, the addition of the decellularized bone particles leads to an increase in cell attachment and mineralization without the addition of exogenous factors, due to the modifications in surface topography of the porous bone substitute material and/or the release of calcium ion.

Advantageously, the porous bone substitute material according to the invention consists solely of:
at least one elastomer matrix comprising an elastomer based on poly(caprolactone-urea-urethane), and
particles of decellularized bone.

Advantageously, the porous bone substitute material according to the invention consists of:
at least one elastomer matrix comprising an elastomer based on poly(caprolactone-urea-urethane), and
particles of decellularized bone.

Advantageously, the porous bone substitute material according to the invention comprises:
at least one elastomer matrix comprising an elastomer based on poly(caprolactone-urea-urethane), and
particles of decellularized bone, said particles of decellularized bone having been obtained from a natural bone of human or animal origin or from a synthetic bone.

Advantageously, the porous bone substitute material according to the invention consists of:
at least one elastomer matrix comprising an elastomer based on poly(caprolactone-urea-urethane), and
particles of decellularized bone, said particles of decellularized bone having been obtained from a natural bone of human or animal origin or from a synthetic bone.

Advantageously, the porous bone substitute material according to the invention comprises:
at least one porous elastomer matrix comprising an elastomer based on poly(caprolactone-urea-urethane), and
particles of decellularized bone, said particles of decellularized bone having a diameter comprised between 1 nm and 1 mm, advantageously comprised between 300 μm and 400 μm.

Advantageously, the porous bone substitute material according to the invention comprises:
at least one porous elastomer matrix comprising an elastomer based on poly(caprolactone-urea-urethane), and
particles of decellularized bone, said particles of decellularized bone having a diameter comprised between 1 nm and 1 mm, advantageously comprised between 300 μm and 400 μm, said particles of decellularized bone having been obtained from a natural bone of human or animal origin or from a synthetic bone.

In a particular embodiment of the invention, the decellularized bone particles represent at least 10% by weight of the porous bone substitute material. Advantageously, the decellularized bone particles represent at least 11% by weight of the porous bone substitute material, advantageously at least 12%, advantageously at least 13%, advantageously at least 14%, advantageously at least 15%, advantageously at least 16%, advantageously at least 17%, advantageously at least 18%, advantageously at least 18%, preferably at least 19%, advantageously at least 20%, advantageously at least 21%, advantageously at least 22%, advantageously at least 23%, advantageously at least 24%, advantageously at least 25%, advantageously at least 26%, advantageously at least 27%, advantageously at least 28%, advantageously at least 29%, advantageously at least 30%, advantageously at least 31%, advantageously at least 32%, advantageously at least 33%, advantageously at least 34%, advantageously at least 35%, advantageously at least 36%, advantageously at least 37%, advantageously at least 38%, advantageously at least 39%, advantageously at least 40%, advantageously at least 41%, advantageously at least 42%, advantageously at least 43%, advantageously at least 44%, advantageously at least 45%, advantageously at least 46%, advantageously at least 47%, advantageously at least 48%, advantageously at least 49%, preferably at least 50% by weight of the porous bone substitute material. Advantageously, the decellularized bone particles represent between 10% and 50% by weight of the porous bone substitute material. Advantageously, the decellularized bone particles represent between 11% and 50%, advantageously between 12% and 50%, advantageously between 13% and 50%, advantageously between 14% and 50%, advantageously between 14% and 50%, advantageously between 15% and 50%, advantageously between 16% and 50%, advantageously between 17% and 50%, advantageously between 18% and 50%, advantageously between 19% and 50%, advantageously between 20% and 50%, advantageously between 20% and 50%, advantageously between 21% and 50%, advantageously between 22% and 50%, advantageously between 22% and 50%, advantageously between 23% and 50%, advantageously between 23% and 50%, advantageously between 24% and 50%, advantageously between 25% and 50%, advantageously between 26% and 50%, advantageously between 27% and 50%, advantageously between 27% and 50%, advantageously between 28% and 50%, advantageously between 29% and 50%, advantageously between 30% and 50%, advantageously between 31% and 50%, advantageously between 32% and 50%, advantageously between 33% and 50%, advantageously between 34% and 50%, advantageously between 35% and 50%, advantageously between 35% and 50%, advantageously between 36% and 50%, advantageously between 37% and 50%, advantageously between 38% and 50%, advantageously between 38% and 50%, advantageously between 39% and 50%, advantageously between 40% and 50% by weight of the porous bone substitute material.

In a particularly advantageous embodiment of the invention, the decellularized bone particles represent 33% by weight of the porous bone substitute material. In another particularly advantageous embodiment of the invention, the decellularized bone particles represent 50% by weight of the porous bone substitute material.

In a particular embodiment of the invention, the porous bone substitute material has a multiscale pore size of between 50 μm and 2000 μm. Within the meaning of the present invention, the terms “pore size” and “pore diameter” may be used interchangeably. By “multi-scale pore size” is meant a variable distribution of pore sizes, which is to say comprising both pores of several microns and pores of smaller sizes, in variable proportions. By way of example, a bone substitute material having a multiscale pore size of between 50 μm and 2000 μm means that the bone substitute material comprises, simultaneously and in the same bone substitute material, pores having variable sizes of between 50 μm and 2000 μm. By way of non-limiting example, a bone substitute material having a multiscale pore size of between 50 μm and 2000 μm means that the bone substitute material comprises both, and in the same bone substitute material, pores having for example a size of 50 μm, pores having a size of 100 μm, pores having a size of 500 μm, pores having a size of 1500 μm, pores having a size of 2000 μm.

Advantageously, the size of the multiscale pores of the porous bone substitute material is between 50 μm and 2000 μm, advantageously between 50 μm and 1500 μm, advantageously between 50 μm and 1000 μm, advantageously between 50 μm and 800 μm, advantageously between 100 μm and 1500 μm, advantageously between 100 μm and 1000 μm, advantageously between 100 μm and 800 μm.

In an advantageous embodiment of the invention, the pores of the bone substitute material have a rough surface.

Within the meaning of the present invention, the term macroporosity is used when the size of the pores is greater than 50 nm, microporosity is used when the size of the pores is less than 2 nm and mesoporosity is used when the size of the pores is between 2 nm and 50 nm. Advantageously, the porous bone substitute material has a macroporosity.

In an advantageous embodiment of the invention, the porous bone substitute material has a total porosity greater than or equal to 60%. Within the meaning of the present invention, the term “total porosity” means the ratio of the volume of the empty spaces of material and of the overall volume of the porous bone substitute material. Advantageously, the total porosity of the porous bone substitute material is greater than or equal to 60%, advantageously greater than or equal to 61%. Advantageously greater than or equal to 62%, advantageously greater than or equal to 63%, advantageously greater than or equal to 64%, advantageously greater than or equal to 65%, advantageously greater than or equal to 66%, advantageously greater than or equal to 67%, advantageously greater than or equal to 68%, advantageously greater than or equal to 69%, advantageously greater than or equal to 70%, advantageously, greater than or equal to 71%, advantageously greater than or equal to 72%, advantageously greater than or equal to 73%, advantageously greater than or equal to 74%, advantageously greater than or equal to 75%, advantageously greater than or equal to 76%, advantageously greater than or equal to 77%, advantageously greater than or equal to 78%, advantageously greater than or equal to 79%, advantageously greater than or equal to 80%, advantageously greater than or equal to 81%, advantageously greater than or equal to 82%, advantageously greater than or equal to 83%, advantageously greater than or equal to 84%, advantageously greater than or equal to 85%, advantageously greater than or equal to 86%, advantageously greater than or equal to 87%, advantageously greater than or equal to 88%, advantageously greater than or equal to 89%, advantageously greater than or equal to 90%, advantageously greater than or equal to 91%, advantageously greater than or equal to 92%, advantageously greater than or equal to 93%, advantageously greater than or equal to 94%, advantageously greater than or equal to 95%, advantageously greater than or equal to 96%, advantageously greater than or equal to 97%, advantageously greater than or equal to 98%, advantageously greater than or equal to 99%. In a particularly advantageous embodiment, the porous bone substitute material has a total porosity greater than or equal to 80%.

Advantageously, the total porosity of the porous bone substitute material is between 60% and 95%, advantageously between 61% and 89%, advantageously between 62% and 88%, advantageously between 63% and 87%, advantageously between 64% and 86%, advantageously between 65% and 85%, advantageously between 66% and 84%, advantageously between 67% and 83%, advantageously between 68% and 82%, advantageously between 68%, preferably between 69% and 81%, advantageously between 70% and 80%. In a particularly advantageous embodiment, the porous bone substitute material has a total porosity of between 70% and 90%.

In a particular embodiment of the invention, the porous bone substitute material has an interconnectivity between the pores of between 60% and 100%. Advantageously, the interconnectivity between the pores is between 65% and 100%, advantageously between 70% and 100%, advantageously between 75% and 100%, advantageously between 80% and 100%, advantageously between 85% and 100%, advantageously between 90% and 100%, advantageously between 91% and 100%, advantageously between 92% and 100%, advantageously between 93% and 100%, advantageously between 94% and 100%, advantageously between 95% and 100%, advantageously between 96% and 100%, advantageously between 97% and 100%, advantageously between 98% and 100%, advantageously between 99% and 100%.

In a particularly advantageous embodiment of the invention, the interconnectivity between the pores is greater than 65%, advantageously greater than 70%, advantageously greater than 75%, advantageously greater than 80%, advantageously greater than 85%, advantageously greater than 90%, advantageously greater than 91%, advantageously greater than 92%, advantageously greater than 93%, advantageously greater than 94%, advantageously greater than 95%, advantageously greater than 96%, advantageously greater than 97%, advantageously greater than 98%, advantageously greater than 99%. In a particularly advantageous embodiment of the invention, the porous bone substitute material has an interconnectivity between the pores of 100%.

In a particularly advantageous embodiment, the porous bone substitute material according to the invention has a multiscale pore size of between 50 μm and 2000 μm, a total porosity of between 60% and 95% and an interconnectivity between the pores of between 60% and 100%. Advantageously, the porous bone substitute material according to the invention has a multiscale pore size of between 50 μm and 2000 μm, a total porosity of between 70% and 85% and an interconnectivity between the pores of 100%.

In a particularly advantageous embodiment, the porous bone substitute material comprising at least one elastomer matrix comprising an elastomer based on poly(caprolactone-urea-urethane) and decellularized bone particles has a multiscale pore size of between 50 μm and 2000 μm, a total porosity of between 60% and 95% and an interconnectivity between the pores of between 60% and 100%. Advantageously, the porous bone substitute material comprising at least one elastomer matrix comprising an elastomer based on poly(caprolactone-urea-urethane) and decellularized bone particles has a multiscale pore size of between 50 μm and 2000 μm, a total porosity of between 70% and 85% and an interconnectivity between the pores of 100%. The porosity of the material, the pore size and their interconnection have a major influence on the ability of the porous bone substitute material to vascularize and resorb progressively.

Thus, due to its total porosity comprised between 70% and 85% and its multi-scale pore size comprised between 50 μm and 2000 μm, the porous bone substitute material comprising at least one elastomer matrix comprising an elastomer based on poly(caprolactone-urea-urethane) and decellularized bone particles is particularly suitable for cell migration and bone formation inside said porous bone substitute material. In addition, the size of the multiscale pores of between 50 μm and 2000 μm and the interconnectivity between the pores of 100% make it possible to regulate angiogenesis and osteogenesis within the porous bone substitute material according to the invention itself. Indeed, the interconnected porous network makes it possible to guide the attachment and cell growth, and therefore the growth of the newly formed bone. Thus, the porous bone substitute material comprising at least one elastomer matrix comprising an elastomer based on poly(caprolactone-urea-urethane) and decellularized bone particles therefore allows the migration of progenitor cells and their differentiation into osteoblasts, which makes it an osteoconductive material.

The size of the porous bone substitute material depends on the size and thickness of the bone to be reconstructed. In a particular embodiment of the invention, the porous bone substitute material has a size of between 10 mm and 20 cm and a thickness of between 100 μm and 4 cm. Advantageously, the size of the porous bone substitute material is between 10 mm and 20 cm, advantageously between 50 mm and 20 cm, advantageously between 100 mm and 20 cm, advantageously between 500 mm and 20 cm, advantageously between 1 cm and 20 cm, advantageously between 2 cm and 20 cm, advantageously between 3 cm and 20 cm, advantageously between 4 cm and 20 cm, advantageously between 5 cm and 20 cm, advantageously between 6 cm and 20 cm, advantageously between 7 cm and 20 cm, advantageously between 8 cm and 20 cm, advantageously between 9 cm and 20 cm, advantageously between 10 cm and 20 cm, advantageously between 11 cm and 20 cm, advantageously between 12 cm and 20 cm, advantageously between 13 cm and 20 cm, advantageously between 14 cm and 20 cm, advantageously between 15 cm and 20 cm. In a particular embodiment of the invention the size of the porous bone substitute material is 10 cm, in particular when the bone to be reconstructed is a long bone.

Advantageously, the thickness of the porous bone substitute material is between 100 μm and 4 cm, advantageously between 200 μm and 4 cm, advantageously between 500 μm and 4 cm, advantageously between 1 mm and 4 cm, advantageously between 1 cm and 4 cm, advantageously between 1 cm and 3 cm. In a particular advantageous embodiment, the thickness of the porous bone substitute material is between 1 cm and 3 cm, in particular when the bone to be reconstructed is a long bone. In another particular advantageous embodiment, the thickness of the porous bone substitute material is between 100 μm and 1 cm, in particular when the bone to be reconstructed is a flat bone.

In a particular embodiment of the invention, the porous bone substitute material has a volume of at least 0.1 cm³. Advantageously, the porous bone substitute material has a volume of at least 0.2 cm³, advantageously at least 0.3 cm³, advantageously at least 0.4 cm³, advantageously at least 0.5 cm³, advantageously at least 0.6 cm³, advantageously at least 0.7 cm³, advantageously at least 0.8 cm³, advantageously at least 0.9 cm³, advantageously at least 1 cm³, advantageously at least 2 cm³, advantageously at least 3 cm³, advantageously at least 4 cm³, advantageously at least 5 cm³, advantageously at least 6 cm³, advantageously at least 7 cm³, advantageously at least 8 cm³, advantageously at least 9 cm³, advantageously at least 10 cm³, advantageously at least 20 cm³, advantageously at least 30 cm³, advantageously at least 40 cm³, advantageously at least 50 cm³, advantageously at least 60 cm³, advantageously at least 70 cm³, advantageously at least 80 cm³, advantageously at least 90 cm³, advantageously at least 100 cm³, advantageously at least 150 cm³, advantageously at least 200 cm³, advantageously at least 250 cm³, advantageously at least 300 cm³, advantageously at least 350 cm³, advantageously at least 400 cm³. In an advantageous embodiment, the porous bone substitute material has a volume of between 0.1 and 400 cm³.

In one embodiment of the invention, the bone substitute material may have various forms, advantageously cylindrical, planar or prismatic forms. Advantageously, the bone substitute material may be in the form of a flexible porous sponge, in the form of a flexible porous membrane, in the form of a flexible porous film.

In a particular embodiment of the invention, the bone substitute material according to the invention is used alone. In another embodiment of the invention, the bone substitute material can further be used in combination with an active agent. Advantageously, the active agent is arranged inside the pores of the bone substitute material according to the invention, partially or entirely covering the pores of the bone substitute material. Advantageously, the active agent may be added by one of the following methods: covering the bone substitute material with the active agent, immersing the bone substitute material in the active agent, spraying the active agent onto the bone substitute material, spraying the active agent onto the bone substitute material or any other technique well known to a person skilled in the art making it possible to fill and/or fill in the pores of said bone substitute material. Advantageously, the active agent may be any therapeutic or pharmaceutically active agent (including, but not limited to, nucleic acids, proteins, lipids, and carbohydrates) that has desirable physiological characteristics for application onto the site of implantation. Therapeutic agents include, but are not limited to, anti-infective agents such as antibiotics and antiviral agents; chemotherapeutic agents (e.g., anticancer agents); anti-rejection agents; analgesics and analgesic combinations; anti-inflammatory agents; hormones such as steroids; growth factors (including, but not limited to, cytokines, chemokines and interleukins), coagulation factors (factors VII, VIII, and the like, IX, X, XI, XII, V), albumin, fibrinogen, von Willebrand factor, thrombin inhibitors, antithrombogenic agents, thrombolytic agents, fibrinolytic agents, vasospasm inhibitors, calcium channel inhibitors, vasodilators, and vasospasm inhibitors, anti-hypertensive agents, antimicrobial agents, antibiotics, surface glycoprotein receptor inhibitors, antiplatelet agents, antimitotics, microtubule inhibitors, anti-platelet agents, antimitotics, and the like, microtubule inhibitors, actin inhibitor antisecretory agents, remodelling inhibitors, antisense nucleotides, anti-metabolites, antiproliferative agents, anticancer chemotherapeutic agents, anti-inflammatory steroids, non-steroidal anti-inflammatory agents, immunosuppressive agents, growth hormone antagonists, growth factors, dopamine agonists, radiotherapeutic agents, peptides, proteins, enzymes, extracellular matrix components, angiotensin converting enzyme (ACE) inhibitors, free radical scavengers, chelators, and inhibitors of angiotensin converting enzyme (ACE), antioxidants, antipolymerases, antiviral agents, photodynamic therapy agents and gene therapy agents and other naturally occurring or genetically modified proteins, polysaccharides, glycoproteins and lipoproteins, or a combination thereof, the list not being limiting. In a particularly advantageous embodiment of the invention, the active agent is a combination of therapeutic agents, and in particular a combination of antibiotics and growth factors.

Another aspect of the invention relates to the porous bone substitute material according to the invention for use in bone repair. Within the meaning of the present invention, the term “bone repair” is intended to mean the reconstruction by induction of osteogenesis of a damaged bone. The porous bone substitute material according to the invention may be useful for repairing a variety of orthopaedic lesions. Advantageously, the porous bone substitute material according to the invention can be used for repairing a bone cavity defect and/or repairing a segmental bone defect. Advantageously, the porous bone substitute material according to the invention can be used to repair a bone cavity defect. Advantageously, the porous bone substitute material according to the invention can be used for the repair of a segmental bone defect. Advantageously, the porous bone substitute material according to the invention can be used to repair a maxillo-facial bone defect.

For the purposes of the present invention, the term “cavitary bone defect” means a loss of bone having a volume of at least 0.1 cm³ without loss of continuity relative to the total surface of the bone not having a defect. Advantageously, the loss of bone without loss of continuity has a volume of at least 0.1 cm³. Advantageously, the porous bone substitute material has a volume of at least 0.2 cm³, advantageously at least 0.3 cm³, advantageously at least 0.4 cm³, advantageously at least 0.5 cm³, advantageously at least 0.6 cm³, advantageously at least 0.7 cm³, advantageously at least 0.8 cm³, advantageously at least 0.9 cm³, advantageously at least 1 cm³, advantageously, a volume of at least 2 cm³, advantageously a volume of at least 3 cm³, advantageously at least 4 cm³. Preferably at least 5 cm³, advantageously at least 6 cm³, advantageously at least 7 cm³, advantageously at least 8 cm³, advantageously at least 9 cm³, advantageously at least 10 cm³. Advantageously at least 20 cm³, advantageously at least 30 cm³, advantageously at least 40 cm³. Preferably at least 50 cm³, advantageously at least 60 cm³, advantageously at least 70 cm³. Preferably at least 80 cm³, advantageously at least 90 cm³, advantageously at least 100 cm³. Advantageously at least 150 cm³, advantageously at least 200 cm³, advantageously at least 250 cm³. Advantageously at least 300 cm³, advantageously at least 350 cm³, advantageously at least 400 cm³. In an advantageous embodiment, the loss of bone without loss of continuity has a volume of between 1 and 400 cm³ with respect to the total surface area of the bone not having a defect.

Within the meaning of the present invention, the term “segmental bone defect” is intended to mean a loss of bone along the length of the bone of at least 10 mm with loss of continuity relative to the bone having no defect.

Advantageously, the loss of bone with loss of continuity is of at least 10 mm, advantageously at least 50 mm, advantageously at least 100 mm, advantageously at least 500 mm, advantageously at least 1 cm, advantageously at least 2 cm, advantageously at least 3 cm, advantageously at least 4 cm, advantageously at least 5 cm, advantageously at least 6 cm, advantageously at least 7 cm, advantageously at least 8 cm, advantageously at least 9 cm, advantageously at least 10 cm, advantageously at least 11 cm, advantageously at least 12 cm, advantageously at least 13 cm, advantageously at least 14 cm, advantageously at least 15 cm.

Examples of situations where such defects may exist include post trauma with segmental bone loss, surgery of the bone tumor where the bone was excised and following total arthroplasty of the joints (e.g., impaction transplantation, etc.), bone loss due to an infectious disease, congenital defect. The porous bone substitute material according to the invention can be used as a prosthetic bone remodelling implant or substitute, for example in orthopedic surgery, including revisions of the hip, replacement of bone loss, for example in traumatology, remodelling in maxillo-facial surgery or filling in periodontal defects and post-dental extraction alveolitis, including increasing the crests and increasing sinus elevation. The porous bone substitute material according to the invention can thus be used to correct any number of bone defects at a bone repair site.

In a particular embodiment of the invention, the porous bone substitute material according to the invention can be used to repair any type of bone, of human or animal origin. In a particular embodiment, the porous bone substitute material according to the invention can be used to repair a long bone. By way of example of long bone, mention may in particular be made of the humerus, the femur, the tibia, the fibula, the radius and the ulna. In a particular embodiment, the porous bone substitute material according to the invention can be used to repair a short bone. By way of example of short bone, mention may in particular be made of vertebrae, a patella, a carpal bone and a tarsal bone. In a particular embodiment, the porous bone substitute material according to the invention can be used to repair a flat bone. By way of example of flat bone, mention may in particular be made of ribs, the skull bone, the iliac bone, a scapula and the sternum. Advantageously, the porous bone substitute material according to the invention can be used to repair a maxillo-facial bone.

In a particular embodiment of the invention, the bone repair is greater than or equal to 5% by volume of the volume of the bone to be repaired. Advantageously, the bone repair is greater than or equal to 6% by volume of the volume of the bone to be repaired, advantageously greater than or equal to 7%, advantageously greater than or equal to 8%, advantageously greater than or equal to 9%, advantageously greater than or equal to 10%, advantageously greater than or equal to 11%, advantageously greater than or equal to 12%, advantageously greater than or equal to 13%, advantageously greater than or equal to 14%, advantageously greater than or equal to 15%, advantageously greater than or equal to 16%, advantageously greater than or equal to 17%, advantageously greater than or equal to 18%, advantageously greater than or equal to 19% advantageously greater than or equal to 20%, advantageously greater than or equal to 21%, advantageously greater than or equal to 22%, advantageously greater than or equal to 23%, advantageously greater than or equal to 24%, Advantageously greater than or equal to 25%, advantageously greater than or equal to 26%, advantageously greater than or equal to 27%, advantageously greater than or equal to 28%, advantageously greater than or equal to 29%, advantageously greater than or equal to 30%, Advantageously greater than or equal to 31%, advantageously greater than or equal to 32%, advantageously greater than or equal to 33%, advantageously greater than or equal to 34%, advantageously greater than or equal to 35%, advantageously greater than or equal to 36%, advantageously, greater than or equal to 37%, advantageously greater than or equal to 38%, advantageously greater than or equal to 39%, advantageously greater than or equal to 40%, advantageously greater than or equal to 41%, advantageously greater than or equal to 42%, advantageously greater than or equal to 43%, advantageously greater than or equal to 44%, advantageously greater than or equal to 45%, advantageously greater than or equal to 46%, advantageously greater than or equal to 47%, advantageously greater than or equal to 48%, advantageously, greater than or equal to 49%. Advantageously, the bone repair is greater than or equal to 50% by volume of the volume of the bone to be repaired.

In a particularly advantageous embodiment of the invention, the porous bone substitute material according to the invention can be used for bone repair in humans or animals. By way of example, the animal may be a horse, a pony, a dog, a cat, a rat, a mouse, a pig, a sow, a cow, a beef, a bull, a calf, a goat, a sheep, a ram, a lamb, a donkey, a camel, a dromedary, the list not being limiting.

Another aspect of the invention relates to a bone repair kit comprising the porous bone substitute material according to the invention and a fixing element. Advantageously, the bone repair kit comprises the porous bone substitute material comprising at least one elastomer matrix comprising an elastomer based on poly(caprolactone-urea-urethane) and decellularized bone particles according to the invention and a fixing element. Within the meaning of the present invention, the term “fixing element” is intended to mean a metal plate or a tubular, circular, internal or external fixing device intended for fixing the metal plate in order to maintain the porous bone substitute material according to the invention in place on the bone to be repaired until it consolidates. Advantageously, the fixator may be a locked plate with screw holes provided with a thread, enabling counter-screws to be put in place, preventing screws from moving backwards, such as the Surfix® fixator. In another embodiment, the fixative may be a Polyetheretherketone (PEEK) plate from the RiSystem company or a steel plate.

Another aspect of the invention relates to a method for preparing a porous bone substitute material according to the invention. In a particular embodiment of the invention, the porous bone substitute material according to the invention is obtained by the poly-HIPE method (formation and polymerization/crosslinking of emulsions with a high internal phase). The high internal phase emulsion or HIPE consist of immiscible liquid/liquid dispersed systems, in which the volume of the internal phase, also called dispersed phase, occupies a volume greater than about 74-75% of the total volume of the emulsion, that is to say a volume greater than that which is geometrically possible for the compact packing of monodisperse spheres.

In a particular embodiment, the method for preparing a porous bone substitute material comprises the following steps:
a) preparing an organic phase comprising the compounds necessary for the synthesis of poly(ester-urea-urethane),
b) adding water and the decellularized bone particles to the organic phase of step a) to form an emulsion,
c) polymerizing/crosslinking the emulsion containing the decellularized bone particles of step c) to obtain said porous bone substitute material,
d) washing said porous bone substitute material obtained at step c), and
e) drying said porous bone substitute material obtained in step d).

In one embodiment of the invention, step a) consists in preparing an organic phase comprising the compounds necessary for the synthesis of the poly(ester-urea-urethane). Advantageously, the organic phase further comprises an oligoester, an organic solvent of the oligoester, a crosslinking agent, a catalyst and a surfactant. Advantageously, the organic phase comprises the toluene, the polycaprolactone triol oligomer, the Span80 surfactant, the hexamethylene diisocyanate crosslinking agent (HMDI) and the dibutyltin dilaurate catalyst (DBTDL). In a particular embodiment, step a) comprises a first step a 1) consisting in solubilizing in toluene, the polycaprolactone triol oligomer and the Span80 surfactant, then a second step a 2) consisting in adding the HMDI crosslinking agent and the DBTDL catalyst to the solution of step a 1) to form the organic phase. In an advantageous embodiment of the invention, 7 ml of toluene is used, 1.3 g of polycaprolactone triol oligomer, 1.3 g of Span80 surfactant, 1.04 ml of HMDI crosslinking agent and 12 drops of DBTDL catalyst are used. Advantageously, a person skilled in the art will know how to adapt the amounts of toluene, of polycaprolactone triol oligomer, of Span80 surfactant, of HMDI crosslinking agent and of DBTDL catalyst as a function of the desired pore size for the porous bone substitute material.

In a particular embodiment, step b) of the method consists in adding the water to the organic phase to form the emulsion, then in adding decellularized bone particles to the emulsion. Advantageously, the water and the decellularized bone particles are introduced gradually and concomitantly with stirring, until an emulsion is obtained. Advantageously, the water is sterilized distilled water. Advantageously, a person skilled in the art will know how to adapt the amount of water as a function of the size of the pores desired for the porous bone substitute material. Advantageously, the amount of water added is 34 mL.

In a particular embodiment, step c) of the process consists in polymerizing/crosslinking the emulsion obtained in step b) in order to obtain said porous bone-substitute material. Advantageously, the polymerization/crosslinking is carried out in a mold in order to give the porous bone-substitute material the desired shape. Advantageously, the emulsion obtained in step b) is placed at a temperature of between 30° C. and 80° C. for 10 to 30 hours. Advantageously, the emulsion obtained in step b) is placed at a temperature of between 35° C. and 65° C., advantageously at a temperature of between 40° C. and 60° C. advantageously at a temperature of between 45° C. and 65° C., advantageously at a temperature of between 50° C. and 60° C., advantageously at a temperature of 55° C. Advantageously, the emulsion obtained in step b) is placed at a temperature of between 30° C. and 80° C. for 10 to 30 hours, advantageously for 11 to 29 hours, advantageously for 12 to 29 hours, advantageously for 13 to 28 hours, advantageously for 14 to 27 hours, advantageously for 15 to 27 hours, advantageously for 16 to 27 hours, advantageously for 17 to 27 hours, advantageously for 18 to 26 hours, advantageously for 19 to 25 hours, advantageously for 20 to 24 hours, advantageously for 22 hours. Advantageously, a person skilled in the art will know how to adjust the temperature according to the desired pore size for the porous bone substitute material.

In a particular embodiment of the invention, the porous bone substitute material obtained at step c) is annealed prior to step d). Advantageously, the porous bone substitute material obtained at step c) is annealed for at least 1 hour at a temperature of at least 50° C. Advantageously, the porous bone substitute material obtained in step c) is annealed for 2 hours at a temperature of 100° C.

In a particular embodiment, the washing step of step d) makes it possible to remove the reagents required for the synthesis of the poly(ester-urea-urethane) which have not reacted during the polymerization, as well as the surfactant and the catalyst which are still present. Advantageously, the washing of step d) is carried out using one of the following products: dichloromethane, dichloromethane/hexane, hexane, water, a mixture of these products or the successive application of these products. Advantageously, the washing of step d) is carried out by bringing the dried porous bone substitute material into contact with dichloromethane for at least 24 hours, followed by a washing step with dichloromethane/hexane (50% vol/50% vol) for at least 24 hours, followed by a washing step with hexane for at least 24 hours, then a last washing with distilled water for at least 24 hours.

In a particular embodiment, the method according to the invention may further comprise a drying step between step c) and step d). Advantageously, this drying step can be carried out by drying in open air or in an oven. A person skilled in the art will know how to adjust the temperature of the oven as a function of the material to be dried. Advantageously, the drying is carried out by open air drying for at least 7 days.

In a particular embodiment, the drying of step c) may be carried out by drying in open air or in an oven. A person skilled in the art will know how to adjust the temperature of the oven as a function of the material to be dried. Advantageously, the drying is carried out by open air drying for at least 15 days.

In a particular embodiment, the method according to the invention may further comprise a sterilization step f) after the step c) of washing said porous bone substitute material. In a particular embodiment, the sterilization step f) can be carried out directly on the dry porous bone substitute material or after a vacuum washing of the biomaterial in an aqueous medium. Advantageously, the sterilization is carried out after a vacuum washing in an aqueous medium.

In one embodiment, the sterilization step f) is carried out as follows:
f1) contacting the porous bone substitute material in sterile water for one hour under vacuum,
f2) replacing the sterile water and contacting the porous bone substitute material in the replacing sterile water for 4 hours under vacuum,
f3) contacting the porous bone substitute material from step f2) in 70% ethanol for 1 hour under vacuum,
f4) replacing the 70% ethanol with sterile water and contacting the porous bone substitute material from step f3) in the sterile water, overnight at ambient pressure,
f5) sterilizing the biomaterial according to the invention resulting from step f4) in water in an autoclave.

In another embodiment, the sterilization step f) can be carried out by gamma radiation. In another embodiment, the sterilization step f) can be carried out by beta radiation. Advantageously, the dose of beta and/or gamma radiation may be between 15 and 45 kGy. Advantageously, the dose of beta and/or gamma radiation is 25 kGy. Advantageously, the dose of beta and/or gamma radiation is 15 kGy.

In another embodiment, the sterilization step f) can be carried out by bringing the bone substitute material into contact with ethylene oxide.

In another embodiment, the sterilization step f) can be carried out by bringing the bone substitute material into contact with a plasma phase derived from a gas.

In another embodiment, the sterilization step f) can be carried out by irradiating the bone substitute material with an electron beam (E-beam, Faisceau E). The electron beam irradiation treatment has the following advantages: shorter treatment duration, improvement of the efficiency of the supply line, less risk of weakening of the elastomer matrix, less oxidative damages in the biomaterial, absence of colour change of the elastomer matrix, making it clean and safe. In addition, the electron beam irradiation treatment is an ecological treatment.

In a particular embodiment, the method according to the invention may further comprise a step g) of preserving said porous bone substitute material after the sterilization step e). Advantageously, step g) of preserving said storage material is carried out by bringing the porous bone substitute material into contact in 70% ethanol until its use.

In a particular embodiment of the invention, the method for preparing a porous bone substitute material comprising the following steps:
a) preparing an organic phase comprising the compounds necessary for the synthesis of poly(ester-urea-urethane),
b) adding water and the decellularized bone particles to the organic phase of step a) to form an emulsion,
c) polymerizing/crosslinking the emulsion obtained at step b) to obtain said porous bone substitute material, and
d) washing said porous bone substitute material obtained at step c),
e) drying said porous bone substitute material obtained at step d),
f) sterilizing the porous bone substitute material obtained at step e), and
g) optionally, storing the porous bone substitute material.

In a particular embodiment of the invention, the method for preparing a porous bone substitute material comprising the following steps:
a) preparing an organic phase comprising the compounds necessary for the synthesis of poly(ester-urea-urethane),
b) concomitantly adding water and the decellularized bone particles to the organic phase of step a) to form an emulsion,
c) polymerizing/crosslinking the emulsion obtained at step b) to obtain said porous bone substitute material, and
d) washing said porous bone substitute material obtained at step c) e) drying said porous bone substitute material obtained at step d)
f) sterilizing the porous bone substitute material obtained at step e), and
g) optionally, storing the porous bone substitute material.

In a particularly advantageous embodiment of the invention, the method for preparing a porous bone substitute material comprising the following steps:
a) preparing an organic phase comprising the compounds necessary for the synthesis of poly(ester-urea-urethane), said step a) comprising a first step a1) consisting in solubilizing, in toluene, the polycaprolactone triol oligomer and the surfactant Span80, then a second step a2) consisting in adding the crosslinking agent HMDI and the catalyst DBTDL to the solution of step a1) to form the organic phase,
b) adding water and the decellularized bone particles to the organic phase of step a) to form an emulsion,
c) polymerizing/crosslinking the emulsion obtained at step b) to obtain said porous bone substitute material, and
d) washing said porous bone substitute material obtained at step c)
e) drying said porous bone substitute material obtained at step d) for at least 15 days,
f) sterilizing the porous bone substitute material obtained at step e) and,
g) optionally, storing the porous bone substitute material.

### FIGURES

FIG. 1 shows the allogeneic bone granules distributed homogeneously inside and on the surface of the porous bone substitute material according to the invention. The images were obtained by 3D microscopy (VHX Keyence) of the porous bone substitute material according to the invention after washing without staining the bone granules, the arrows indicating the presence of bone granules (A, B).

FIG. 2 represents the Fourier transform infrared spectroscopy (FTIR) analysis of the bone granule alone, of the poly(caprolactone-urea-urethane) elastomer matrix alone and of the porous bone substitute material according to the invention (composite).

FIG. 3 shows the stress-deformation curves of the poly(caprolactone-urea-urethane) elastomer matrix alone and of the porous bone substitute material according to the invention (composite) during compression tests (on the left: whole curves; on the right: zoom at the start of the curves).

FIG. 4 represents the loss of mass of the poly(caprolactone-urea-urethane) elastomer matrix alone and of the porous bone substitute material according to the invention (composite) during the in vitro degradation at 37° C. and accelerated at 90° C.

FIG. 5 represents the cell activity determined by MTT test after incubation with the extraction media of the poly(caprolactone-urea-urethane) elastomer matrix alone and of the porous bone substitute material according to the invention (composite). “Blanc” (Blank sample) represents the result for control cells under normal conditions, and “contrôle positif” (positive control) represents the result for cells in the presence of a cytotoxic molecule (here selected as HMDI).

FIG. 6 represents the cell viability determined by trypan blue staining during an indirect cytotoxicity test of the poly(caprolactone-urea-urethane) elastomer matrix alone and of the porous bone substitute material according to the invention (composite). “Blanc” (Blank sample) represents the result for control cells under normal conditions, and “contrôle positif” (positive control) represents the result for cells in the presence of a cytotoxic molecule (here selected as HMDI).

FIG. 7 represents the migration of mesenchymal stromal cells obtained from canine adipose tissues from day 10 (J 10) to day 40 (J 40) within the poly(caprolactone-urea-urethane) elastomer matrix alone and from the porous bone substitute material according to the invention (composite).

FIG. 8 shows a segmental defect of critical size on a rat femur.

FIG. 9A represents the cylinders of the porous bone substitute material according to the invention before implantation in a packaging medium.

FIG. 9B represents the cylinders of the porous bone substitute material according to the invention after microtomography acquisition.

FIG. 10A represents the radiological monitoring of a segmental femoral defect held by an osteosynthesis plate, with partial reconstruction after 61 days.

FIG. 10B represents the radiological monitoring of a segmental femoral defect held by an osteosynthesis plate, with the appearance of a failure of the system after 31 days.

FIG. 11 represents the concentrations of red blood cells and blood platelets for the control batches (Non operated (Non op)), Empty Control (“Témoin vide”), poly(caprolactone-urea-urethane) elastomer matrix alone (“Elastomère”), bone substitute material according to the invention (Composite), decellularized bone and positive control (Cpos) (noncritical) at the various study times.

FIG. 12 represents the concentrations of white blood cells and lymphocytes for the control batches (non operated (Non op)). Empty Control (“Témoin vide”), elastomer matrix Poly(caprolactone-urea-urethane) alone (“Elastomère”), bone substitute material according to the invention (Composite), decellularized bone and positive control (C pos) (noncritical) at the different study times.

FIG. 13 represents the concentrations of serum markers of bone metabolism (CTX: bone resorption; P1PN and Oc: bone synthesis) for the control batches (Non operated (Non op)), Empty Control (“Témoin vide”), poly(caprolactone-urea-urethane) elastomer matrix alone (Elastomer), bone substitute material according to the invention (Composite), decellularized bone and positive control (C pos) (non-critical) at the various study times.

FIG. 14A represents the quantity of bone formed after 1 or 3 months inside the area of the bone defect for the control batches (Non operated (Non Op)), Empty Control (“Témoin vide”), poly(caprolactone-urea-urethane) elastomer matrix alone (“Elastomère”), bone substitute material according to the invention (Compo) and positive control (Cpos) (non-critical) at the various study times.

FIG. 14B represents the bone quantity/initial volume ratio of the bone defect for the control batches (on operated), Empty Control (“Témoin vide”), poly(caprolactone-urea-urethane) elastomer matrix alone (“Elastomère”), bone substitute material according to the invention (Compo) and positive control (C pos) (non-critical) at the various study times.

FIG. 15A represents the surface area of the bone formed after 1 or 3 months inside the area of the bone defect for the control batches (on operated), Empty Control (“Témoin vide”), poly(caprolactone-urea-urethane) elastomer matrix alone (“Elastomère”), bone substitute material according to the invention (Compo) and positive control (C pos) (noncritical) at the various study times.

FIG. 15B represents the bone surface/initial volume ratio of the bone defect for the control batches (on operated), Empty Control (“Témoin vide”), poly(caprolactone-urea-urethane) elastomer matrix alone (“Elastomère”), bone substitute material according to the invention (Compo) and positive control (C pos) (noncritical) at the various study times.

FIG. 16 represents the bone defect remained empty after 3 months for the “Control” batch, ((A): top view in 3D-microtomography and (B): side view in 3D-microtomography).

FIG. 17A represents the bone defect of non-critical size for the “contrôle positif” (“positive control”) batch, in top view in 3D microtomography after 3 months.

FIG. 17B represents the bone defect of non-critical size for the “contrôle positif” (“positive control”) batch, in a side view in 3D microtomography after 3 months.

FIG. 17C represents the bone defect of non-critical size for the “contrôle positif” (“positive control”) batch, in histological section with Masson trichrome staining (magnification×5) after 1 month.

FIG. 17D represents the detail of the central area of the bone defect for the “positive control” batch (image insert C, magnification×40).

FIG. 18A represents the bone defect of critical size for the poly(caprolactone-urea-urethane) elastomer matrix batch alone, in a top view in 3D-microtomography after 1 month.

FIG. 18B represents the bone defect of critical size for the poly(caprolactone-urea-urethane) elastomer matrix batch alone, in a top view in 3D-microtomography after 3 months.

FIG. 18C represents the bone defect of critical size for the poly(caprolactone-urea-urethane) elastomer matrix batch alone, in histological section with Sudan Black staining (magnification×2, 5) after 1 month.

FIG. 18D represents the detail of the central zone of the bone defect for the batch of poly(caprolactone-urea-urethane) elastomer matrix alone (image insert C, magnification×40), with a Masson trichrome coloration after 1 month.

FIG. 19 represents the bone defect after 3 months for the poly(caprolactone-urea-urethane) elastomer matrix batch alone ((A): side view in 2D-microtomography, (B): side view in 3D-microtomography and (C): bottom view in 3D-microtomography).

FIG. 20 represents the bone defect after 3 months for the “decellularized bone” batch, in a side view in 3D-microtomography.

FIG. 21A represents the bone defect of critical size for the batch of porous bone substitute material according to the invention “composite”, in a top view in 3D-microtomography after 1 month.

FIG. 21B represents the bone defect of critical size for the batch of porous bone substitute material according to the invention “composite”, in a top view in 3D-microtomography after 3 months.

FIG. 21C represents the bone defect of critical size for the batch of porous bone substitute material according to the invention “Composite”, in histological section with a Masson trichrome coloration after 1 month (magnification×2, 5).

FIG. 21D represents the detail of the central area of the bone defect for the batch of porous bone substitute material according to the invention “composite” (image insert C, magnification×40).

FIG. 21E represents the bone defect of non-critical size for the batch of porous bone substitute material according to the invention “Composite”, in histological section with a staining with Masson trichrome after 1 month (magnification×2, 5).

FIG. 21F represents the detail of the outer zone of the bone defect for the batch of porous bone substitute material according to the invention “composite” (image insert C, magnification×40).

### EXAMPLES

### Example 1: Formulation and Synthesis of the Porous Bone Substitute Material According to the Invention

First, the Allodyn® allogeneic material (decellularized bone) was ground so as to produce granules with a diameter ranging from 50 to 500 μm. In order to obtain particles of controlled particle size, the mixture of granules obtained after grinding is sieved using a screening machine (Fisher AS 200 TAP). Thus, the granules could be separated according to granulometries of between 50 and 100 μm, 100 to 200 μm, 200 to 300 μm and 300 to 400 μm. The granules larger than 400 μm were not retained here.

Second, these granules were incorporated into the elastomer matrix comprising an elastomer based on poly(caprolactone-urea-urethane) during the synthesis of the latter by poly-HIPE method (formation and polymerization/crosslinking of high internal phase emulsions). Several allogeneic elastomer matrix/bone ratios were tested.

These steps have been validated for a source of human decellularized allogeneic bone: Allodyn®. For reasons of availability in sufficient quantity during the study, a source of decellularized xenogeneic bone of bovine origin, Laddec®, was also tested without observing the slightest variation.

The retained compositions used are:
bone substitute material A, with 100% allogeneic elastomer/bone matrix (1 g/1 g) ratio, namely a bone mass fraction of 50% and grain size between 300 and 400 m,
Bone substitute material B, with 50% allogeneic elastomer/bone matrix ratio (1 g/0, 5 g), i.e., a mass bone fraction of 33% and grain size comprised between 300 and 400 μm.

A higher proportion of bone than 50% results in a loss of structure of the bone substitute material.

### Example 2: Physicochemical and Mechanical Properties of the Bone Substitute Material According to the Invention

The physicochemical properties of the bone substitute materials according to the invention were tested by:
Fourier Transform Infrared (FTIR) spectroscopy for analysing chemical functions present in the synthesized substitute materials;
Scanning electron microscopy (SEM) for the morphological observation of the substitute materials, coupled with an elemental analysis (EDX);
Measurement of volumetric absorption rates to determine the interconnectivity of the porous structure;
Alizarin Red Coloration to evaluate and visualize the incorporation of allogeneic bone particles within the substitute material. This dye is a specific marker for calcium deposits.

For clarity, the results detailed below are given for the bone substitute material B having a bone mass fraction of 33%, and comprising bone particles with a diameter of 300 to 400 μm.

### 1. Interconnectivity/Porosity

After washing, the density of the bone substitute material was evaluated by pycnometry.

The found value of 1.29 in comparison with that of the matrix alone (1.05) or the bone alone (2.59) indicates that the bone particles are still present in the elastomer matrix.

A mass fraction of bone of 31% is found after washing, compared to the mass fraction of 33% initially introduced into the emulsion.

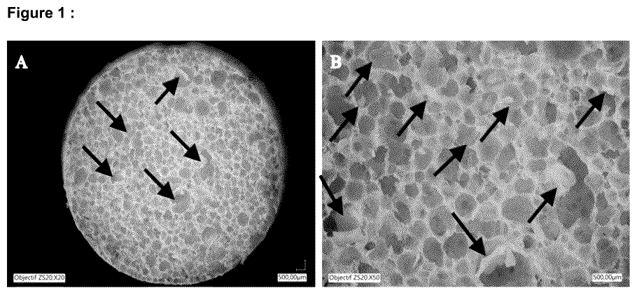

The decellularized bone particles appear to be homogeneously distributed inside and on the surface of the bone substitute material (FIG. 1).

In addition, measurements of the volumetric absorption rate have shown that the interconnectivity of the porous structure has only been very slightly modified by the incorporation of the bone particles and remains greater than 80%.

This value is compatible with good penetration of liquids and cell migration (rv Poly(caprolactone-urea-urethane) elastomer matrix alone=100.8±8% vs. rv Composite=86.4±2%).

### 2. Chemical Composition

TABLE 1
Elementary composition obtained by EDX analysis of decellularized
bone particles alone, poly(caprolactone-urea-urethane) elastomer
matrix alone and bone substitute material according to the invention
(composite) (nitrogen not taken into account for the comparison
with experimental limited by sensitivity of the apparatus).

Mass percentage of elements

| | | | |
|---|---|---|---|
| % C | % O | % P | % Ca |

Theory

| | | | | |
|---|---|---|---|---|
| poly(caprolactone- | 70.78 | 29.22 | — | — |
| urea-urethane) | | | | |
| matrix | | | | |
| Porous bone | 60.2 | 31.0 | 3.1 | 5.7 |
| substitute material | | | | |
| according to the | | | | |
| invention | | | | |
| (composite) | | | | |

Experimental

| | | | | |
|---|---|---|---|---|
| poly(caprolactone | 75.5 ± | 24.5 ± | — | — |
| urea-urethane) | 3.4 | 3.42 | | |
| matrix | | | | |
| Decellularized | 50.5 ± | 32.7 ± | 5.9 ± | 10.9 ± |
| bone particles | 0.4 | 0.5 | 0.1 | 0.1 |
| Porous bone | 65.8 ± | 27.4 ± | 1.5 ± | 5.3 ± |
| substitute material | 1.2 | 1.3 | 0.2 | 0.3 |
| according to the | | | | |
| invention | | | | |
| (composite) | | | | |

The elemental analysis shows a good correlation between the theoretical expected values according to the amount of bone incorporated during the synthesis of the material, and the experimental values of the material after washing. This proves once again the presence of bone particles in the elastomer matrix. The chemical structure of the elastomer matrix has not, as for her, been modified since all the peaks associated with this matrix are found (FIG. 2).

### 2.3. Mechanical Properties

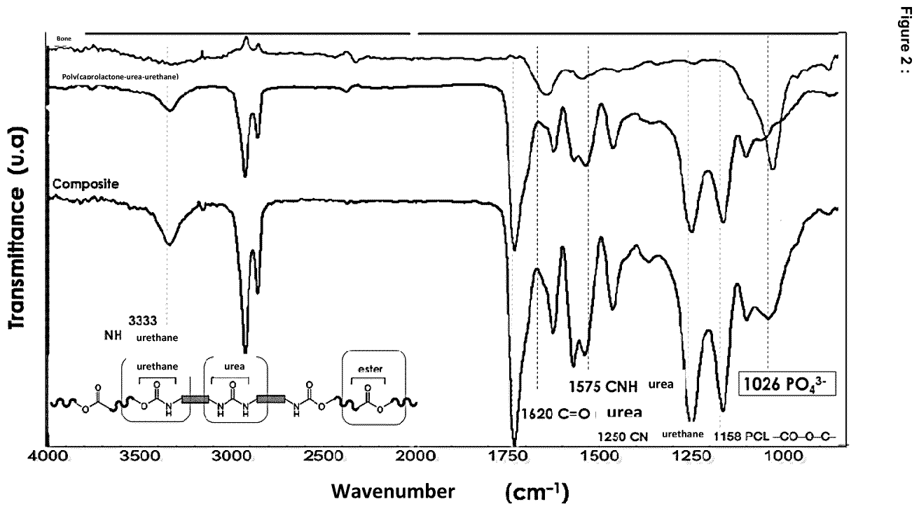

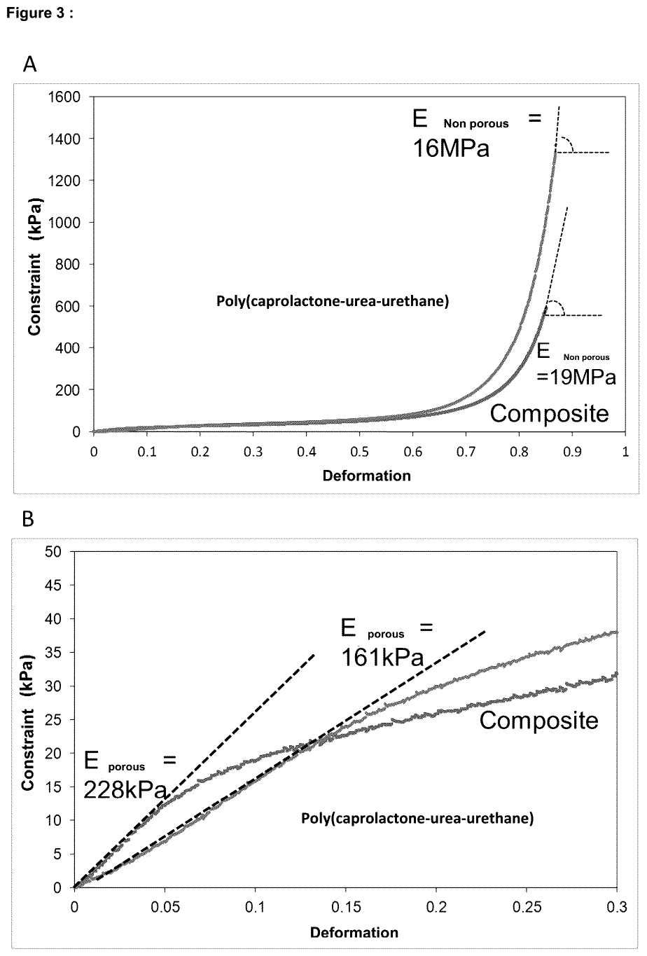

The study of the mechanical properties required the production of samples of larger diameter and the validation of the synthesis steps. Once the latter has been completed, the elastomer nature of the bone substitute material according to the invention and of the poly(caprolactone-urea-urethane) elastomer matrix alone was brought out by mechanical compression tests: the shape of the Constraint curve vs Deformation of the bone substitute material according to the invention is similar to that of the poly(caprolactone-urea-urethane) elastomer matrix alone proving the elastomer nature of the materials (FIG. 3).

The Young's modulus E of the porous material may be defined on the first linear portion of the curve. A value of 228 kPa was found for the bone substitute material according to the invention, which is in the range of elastomer foams (1<E<1000 kPa). In the second linear part of the curve, the Young's modulus E of the non-porous material, when the pores are all crushed, can be defined. A value of 19 MPa was found for the bone substitute material according to the invention. The values are higher than those found for the poly(caprolactone-urea-urethane) elastomer matrix alone. Thus, the decellularized bone particles participate in a slight increase in the moduli of the bone substitute material according to the invention, while retaining the elastomer nature of the polymer matrix.

### 2.4. Degradation Kinetic

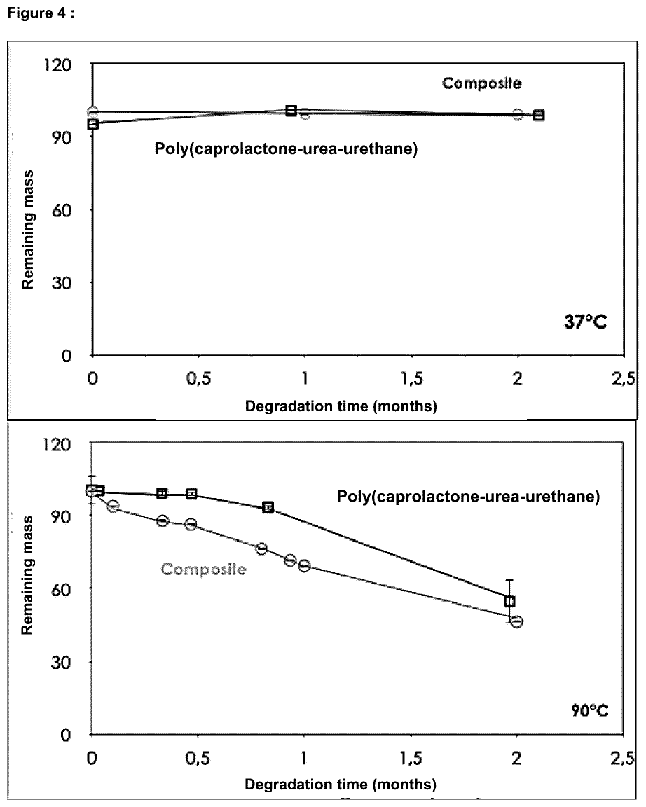

An important criterion during the production of a bone substitute material for tissue engineering is its resorbability since it must be replaced over time by the newly formed bone. It has been suggested that for regeneration of bone tissue, the bone substitute material must have reduced hydrophilicity, such that the degradation rate may exceed 18 months. In vitro degradation studies were carried out according to standards ISO 10993-13. The degradation kinetic was notably evaluated by measuring the mass loss. The accelerated degradation tests at 90° C. showed that the bone substitute material degrades slightly faster than the poly(caprolactone-urea-urethane) elastomer matrix alone (FIG. 4). This is due to an increase in the hydrophilicity of the materials as attested to measurements of angle of contact with water: θ=121±10° for the poly(caprolactone-urea-urethane) elastomer matrix alone vs. θ=83±22° for the bone substitute material.

Thus, the poly(caprolactone-urea-urethane) elastomer matrix alone is stable for 14 days at 90° C. By applying the “rule of ten” giving the relationship between the increase in degradation rate when the temperature is raised by ten degrees and with a Q10 factor of 2-2.5 [ASTM F 1980-02: Standard Guide for Accelerated Aging of Sterile Medical Device Packages], the lifetime of the poly(caprolactone-urea-urethane) elastomer matrix alone at 37° C. is estimated to be between 19.4 and 63.4 months.

For the bone substitute material, the lifetime at 90° C. is 10 days which leads to an estimation of the lifetime at 37° C. between 13.0 and 42.3 months. This stability is suitable for use of the bone substitute material as a support for bone regeneration.

### Example 3: Interactions Between the Porous Bone Substitute Material According to the Invention and Mesenchymal Stromal Cells Obtained from Canine Adipose Tissues (CSM)

### 3.1. Cytotoxicity Dosages

First, the cytotoxicity of extraction products optionally released by the bone substitute material according to the invention was studied in accordance with standards ISO 10993-5 and 10993-12.

Thus, the bone substitute material as obtained in Example 1 and the poly(caprolactone-urea-urethane) elastomer matrix alone are incubated in standard culture medium for 24 h at 37° C. Then this extraction medium is deposited on a carpet of MSC at 80% confluence. After 24 h of incubation, the metabolic activity of the cells is measured by an MTT dosage.

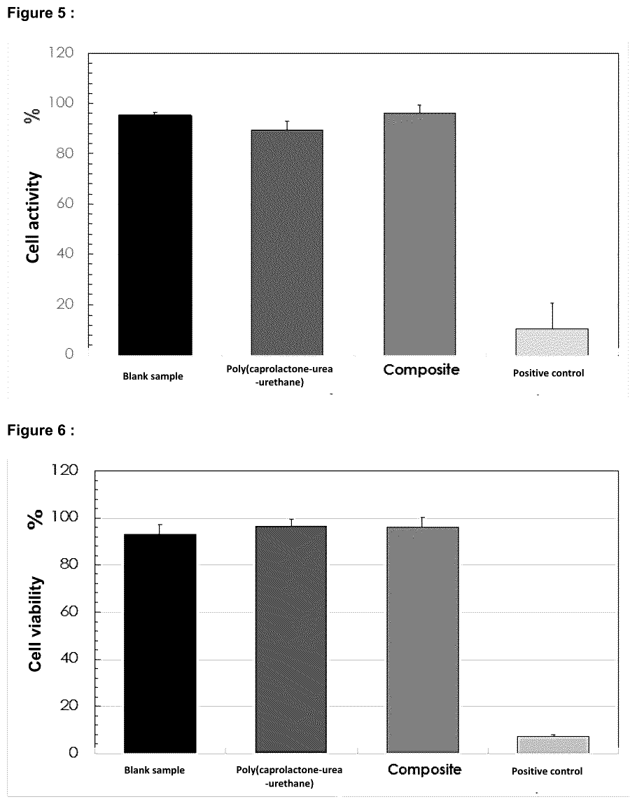

The results are shown in (FIG. 5).

The standard sets a cell viability limit of 70% for a product to be considered as non-cytotoxic.

The results obtained show no difference in metabolic activity between the control cells and those placed in the presence of the extraction medium of the poly(caprolactone-urea-urethane) elastomer matrix alone or of the bone substitute material according to the invention (composite). This metabolic activity is greater than 90%, and therefore at the limit set by the standard. These results show, under these conditions, the absence of cytotoxicity of the extracts of the materials. Second, we evaluated the effect of cytotoxic product release by an indirect cytotoxicity test. To do this, the materials are deposited, without direct contact, above the CSM at 80% confluence. After 24 h of incubation at 37° C. cell viability is measured by trypan blue staining.

The results are presented in (FIG. 6). The cell viability obtained is greater than 80% and comparable between the control and the cells placed in the presence of the poly(caprolactone-urea-urethane) elastomer matrix alone or of the bone substitute material according to the invention (composite). The indirect interaction of scaffold-cells does not generate cytotoxic compounds in the culture medium after 24 hours.

### 3.2. Interactions of CSM Cells and Bone Substitute Materials.

Colonization tests with canine CSM were carried out in order to test the “attraction” power of the bone substitute material according to the invention (composite) and of the poly(caprolactone-urea-urethane) elastomer matrix alone. The bone substitute material according to the invention (composite) and the elastomer matrix poly(caprolactone-urea-urethane) alone are respectively deposited on a bed of CSM at 80% confluence.

Cell migration was determined at J10 (day 10), J20 (day 20), J30 (day 30) and J40 (day 40).

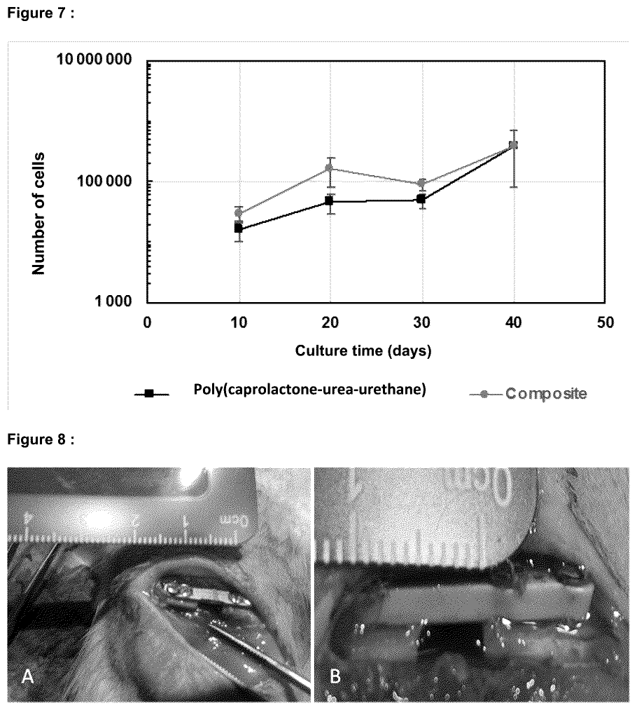

The results are presented in (FIG. 7).

A counting of the cells present on and inside the bone substitute material according to the invention (composite) and the poly(caprolactone-urea-urethane) elastomer matrix alone is carried out after separation of the cells by enzymatic treatment. The results obtained show that the cells are capable of migrating into the materials.

Conclusions: All of these results demonstrate that the bone substitute material according to the invention (composite) is not toxic, that the cells are capable of adhering thereto and of proliferating therein and that, irrespective of the culture conditions, they colonize it even in depth, demonstrating its osteoconduction properties.

### Example 4: In Vivo Study of the Repair Power of the Bone Substitute Material (Composite) on Rat Model of Segmental Defect

The study is based on the reconstruction of a bone defect of segmental type in the rat (Lewis male 7-9 weeks, January breeding). It makes it possible to evaluate the biocompatibility, the biodegradability and the effectiveness of the biomaterials under real conditions, this type of lesion being similar to those frequently encountered in military or civilians victim of attacks or accidents of the road, etc.

The segmented bone defect model consists in removing a section of bone eliminating any continuity between the two bone segments obtained, and not allowing spontaneous repair. The critical size of this bone defect is defined as being equal (at least) to 1.5 to 2 times the diameter of the bone in question. In order to evaluate the effectiveness of the bone substitute material according to the invention (composite) with respect to the poly(caprolactone-urea-urethane) elastomer matrix alone, several batches of animals were monitored up to 3 months post-lesion/implant. The efficiency of bone repair was assessed by microtomography and histology on femurs taken after euthanasia. Animals blood was used for blood counts/formulations and for ELISA (on serum) dosages for markers of bone formation and resorption.

Batches of 6 animals were used for each time, 1 and 3 months, i.e., a total of 60 animals.

The right femur is operated, the left femur serves as a reference.
“Control” batch: empty defect, without implant
batch “poly(caprolactone-urea-urethane) elastomer matrix alone”
batch “bone substitute material according to the invention (composite)” (formula 33% of bone, granules 300-400 μm)
Batch “positive control”: noncritical (empty control (“témoin vide”)) size defect
A “control batch” (Non op.) corresponds to animals that weren't operated but underwent anaesthesia and analgesia.

The rat model of segmental bone defect consists in making a section in the femoral diaphysis that does not allow spontaneous repair. The critical size of this bone defect is here from 5 to 6 mm, a length well established in this rodent model. The bone is cut at the diaphysis and the two bone ends are stabilized by a fixator adapted to the femur of the rat (FIG. 8). This fixator is held on the upper face of the femur by 4 screws of diameter 1.1 mm made of stainless steel (Synthes).

### 5.1. Surgical Procedure

Anaesthesia/Analgesia: anaesthesia Ketamine/Medetomidine: 60/0.5 mg/kg intraparenteral.
Waking up: Atipamezole, 1 mg/kg intramuscular
Analgesia: Buprenorphine 0.05 mg/kg postoperative (waking up followed by 3 times a day for 3 days), subcutaneous (SC).

### Surgical Procedure:

Shearing of the posterior leg+back: Disinfection with betadine; Identification of the outer face of the operated thigh;
Incision of the skin and the aponeurosis of the outer face (20-30 mm) and spreading with a rounded tip surgical chisel of muscle planes to expose the central part of the femur (diaphysis) without vascular injury;
installation of the fixator (steel plate or PEEK, L=23 mm) held on the upper face of the femur using a needle holder;
Drilling of the holes intended to receive the screws with continuous saline irrigation (dry with a diameter of 1 mm): Positioning of the 4 transcortical screws;
Transverse section of the bone with oscillating saw (ConMed) and removal of the bone segment (FIG. 11), the inside of the hole is cleaned with delicate injection of saline solution;
placement of the implant in the bone defect;
bringing together the muscle planes. If necessary, add 2 to 3 separate points (resorbable thread). Closure of skin planes by 5 mm staples;
Local application of betadine;
Waking up, the animals are placed in lateral decubitus on a dry sheet in their cage, on a hot plate;
Post-operative care: subcutaneously analgesia during the wake-up and then for 3 days (3 times a day).

The animals use their member operated as soon as they wake-up. The monitoring of animals is daily: wounds are clean and no external sign of inflammation, no lameness or modification of behaviour has been noted when the osteosynthesis system is stable. Weight curves are similar for all batches throughout the study, with a recovery phase of approximately 15 days after surgery.

### 5.2. Preparation of Implants

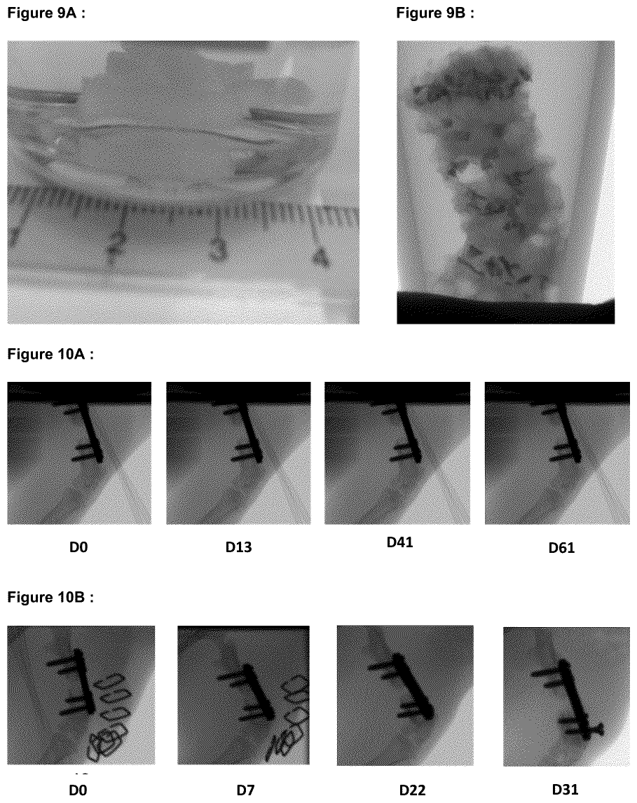

The biomaterials (bone substitute material according to the invention (composite) and the poly(caprolactone-urea-urethane) elastomer matrix alone are cylinders 10 to 15 mm in length and 4 mm in diameter (FIG. 9A). They are prepared the day before implantation in a sterile manner in packaging medium and incubated in an oven at 37° C. and 5% CO₂. They are cut during their installation in the bone defect in order to have dimensions suitable for each animal. Some batches of composites were scanned in microtomography in order to verify the size and homogeneity of the distribution of the granules (FIG. 9B) before the implantation.

The used packaging medium contains DMEM (Dulbecco's Modified Eagle Medium);
Penicillin/Streptomycin 0.8%: Fungizone 1%.

The biomaterials retain their integrity, without breaking up at the time of implantation.

The two types of biomaterials—the poly(caprolactone-urea-urethane) elastomer matrix alone and the porous bone substitute material according to the invention (Composite)—appear to be radio-transparent, which will allow easier radiological monitoring, allowing the newly synthesized bone to be visualized.

Only the Laddec® granules inside the composite are radiopaque and locatable in microtomography (FIG. 9B).

### 5.3. X-Ray Analysis

Radiological monitoring is carried out with an irradiator (SARRP) in imaging mode at 1, 3, 6, 12 days then every 10 to 15 days, and makes it possible to verify the integrity of the osteosynthesis system and the appearance of mineralized bone inside the bone defect (FIG. 10A). In the case of a failure of the osteosynthesis system, as illustrated in (FIG. 10B) where the most distal screw is becoming detached after 31 days, the animals are euthanized and the femurs recovered for analysis.

The qualitative analysis of the images shows no bone formation up to one month post-surgery. At three months, a more or less significant bone callus is visible inside the defect, restoring continuity between the two fragments in half of the animals of the group “bone substitute material according to the invention (composite)”.

### 5.4. Blood Quantitative Assay

Blood is collected from EDTA-K3 by intracardiac puncture of anaesthetized rats in advance of sacrifice, in order to carry out a blood count (Procyte DX-IDEXX veterinary hematology apparatus), in order to detect abnormal inflammation, or a possible effect of biomaterials on the blood formula.

Serum was also prepared from blood collected without anticoagulant and centrifuged at 1500 g for 10 minutes for an ELISA quantitative assay for bone repair markers.

Concerning the red blood cell level, the production remains significant at one and three months compared to the unoperated animals, but no significant variation was detected for each batch and each time. The platelet concentration does not vary (FIG. 11).

The overall level of inflammation is similar for all batches (FIG. 12) and similar to the basic level of unoperated rats, no significant difference has been recorded: the poly(caprolactone-urea-urethane) elastomer matrix alone or the bone substitute material according to the invention (composite) are therefore well tolerated by the organism and do not enhance the inflammatory response due to bone trauma at these late times in this rat model.

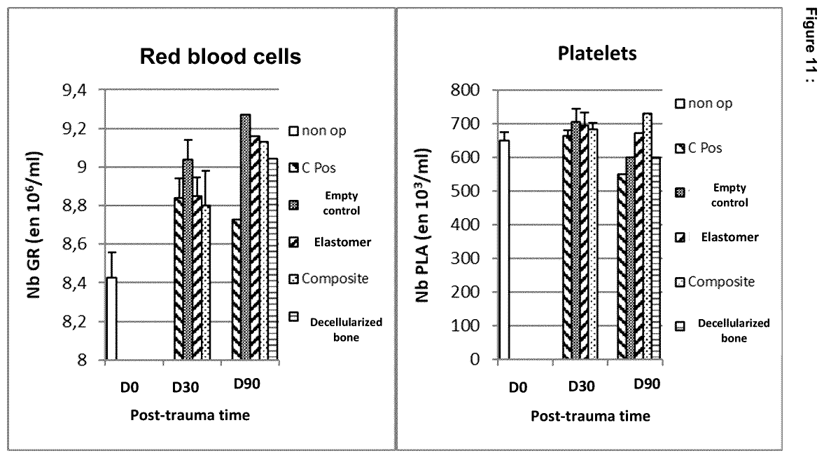

Direct markers of remodelling (FIG. 13) demonstrate a synthesis activity (Oc. P1NP) and a bone resorption activity (CTX1). The interpretation of their quantitative assay is however difficult for animals since their levels can depend, for example, on daytime/night-time cycles. The values observed for P1NP (N-terminal propeptide of type 1 procollagen) instead reflect the phases of osteoblast proliferation. These values are here 4 to 5 times lower than those of the unoperated animals (53.20±2.89 ng/ml). However, a two times higher value is noted for the bone batch decellularized at 3 months (25.52±11.34 ng/ml).

The concentrations of osteocalcin (Oc), serum marker of bone synthesis (reflecting mineralization activity by osteoblasts), remain lower than those of unoperated rats (103 1.35±59.06 ng/ml) for all batches at one month and at three months. It will however be noted that the values of the batches implanted with the various biomaterials at 3 months are similar to those found for the positive controls (625.65±41.15 ng/mL) where complete reconstruction is in progress. These values are 3 times higher than those observed for the control batch (empty defect) for which no reconstruction is observed (221.40±19.50 ng/ml).

All these values would reflect a high activity of the osteoblasts produced when a biomaterial is placed, even if their number remains less than that found in unoperated animals. CTX1 (carboxy-terminal telopeptide of type 1 collagen) is a marker of the level of bone resorption by the cathepsin K pathway, predominant bone remodelling pathway. When the bone defect remains empty (7.89±0.87 ng/ml), its concentration is lower than that of the unoperated animals (9.24±0.52 ng/ml), reflecting a low remodelling activity, linked to a low neosynthesis of bone tissue. This concentration is equivalent to that of the unoperated animals when the poly(caprolactone-urea-urethane) elastomer matrix alone, the porous bone substitute material according to the invention (composite) or the decellularized bone are implanted after one and three months, signifying that remodelling of the newly formed mineralized tissue is in progress.

### 5.5. Microtomographic Analysis

While femurs are fixed in Burckhardt's solution for subsequent histological analyses, they are analysed by microtomography (Skyscan 1174) with the following parameters: 50 kV; 800 mA to visualize and quantify bone neomineralization at segmental defect. Rapid acquisition (40 to 70 min) is carried out on the complete femurs with their fixator with a resolution of 50 to 60 μm, then, depending on the mineralization level and the “solidity” observed at the time of sampling, the fixators are removed and finer acquisition is carried out (14 to 20 μm resolution, 2 to 3 hours) for analysis. The measurement zone corresponds to the zone of the initial bone defect.

The acquired data is processed by a quantification software (CTan, Skyscan), and makes it possible to view and quantify the bone reconstruction at the bone defect level via the BV/TV ratio (bone volume/tissue volume) which translates the quantity of newly formed bone into a given volume.

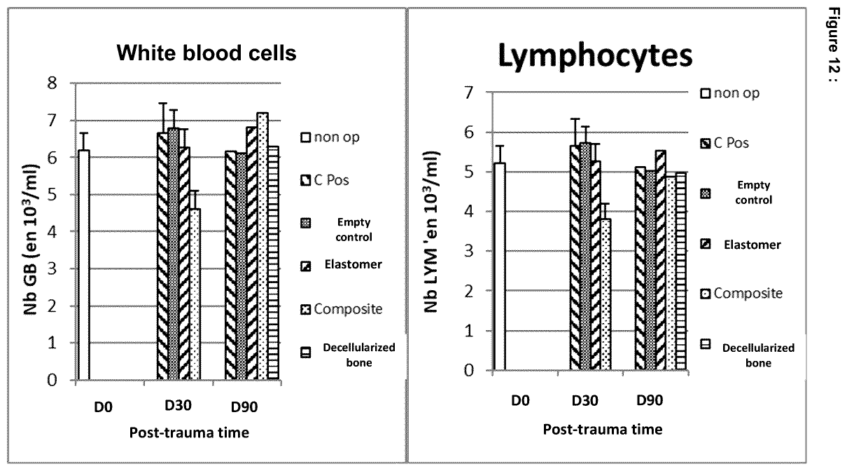

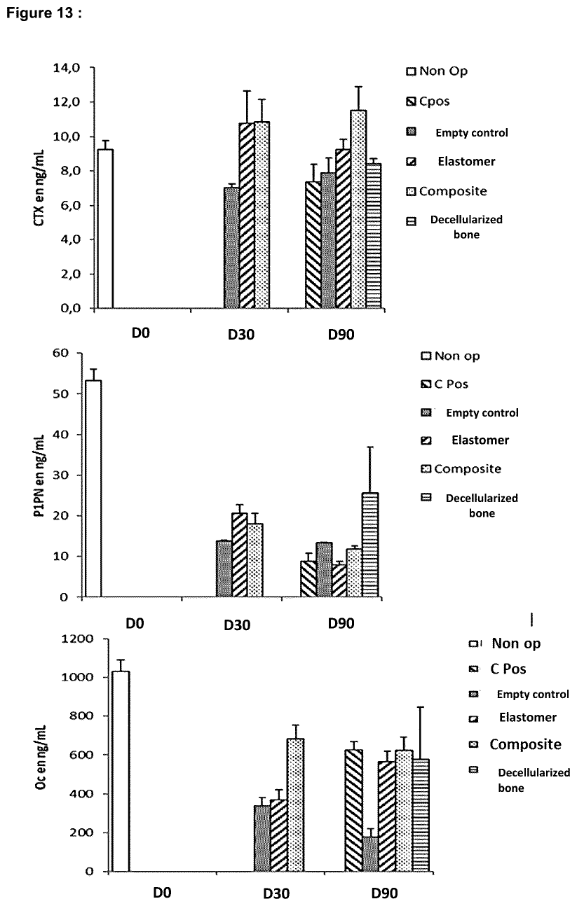

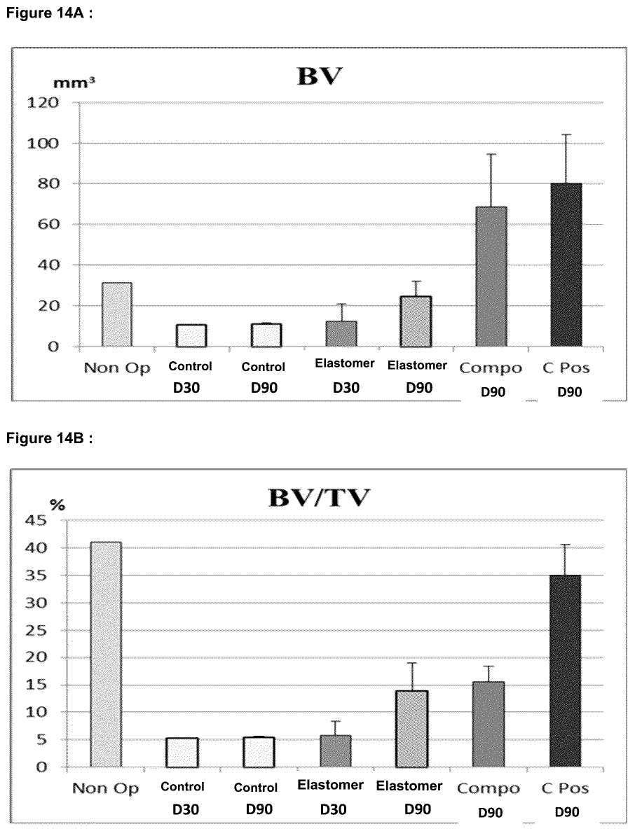

FIG. 14A makes it possible to quantify the bone present in the area of the bone defect. The latter remains 3 times lower at one month and at three months for the control animals compared with the unoperated animals (31.11 mm³), when the defect is left empty. This reflects the absence of bone synthesis and therefore of repair of this model. When the poly(caprolactone-urea-urethane) elastomer matrix alone is implanted, this value is equivalent to that of the control animals at one month (10.8±1, 1 mm³), but is equivalent (24.75±7.29 mm³) to that of the unoperated animals after 3 months.

The installation of the bone substitute material according to the invention (composite) results in a quantity of bone formed at three months 2.5 times greater (68.60±26.02 mm³) than that of the unoperated animals. This value is close to that found for positive controls where the synthesis is here 3 times greater (80.03±23.99 mm³).

However, when this quantity of bone formed is brought back to the initial volume of the zone of the defect (FIG. 14B), the set of values obtained for all the batches remains less than that found for the unoperated animals (41.1). This value reflects the proportion of bone in a defined area and can make it possible to assess the phases of the repair process in progress. In fact, bone repair comprises an active synthesis phase followed by a more or less long remodelling phase of the bone callus. During this last phase, the bone is redesigned and restructured according to the area of the bone in question, here in cortical bone, to allow restoration of the mechanical properties.

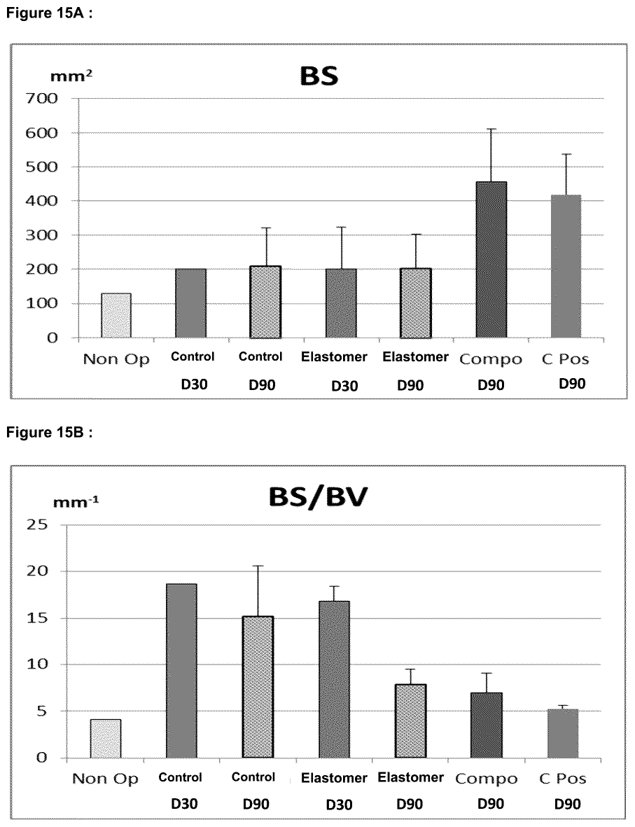

The mirror image of the bone surface ratio (BS) to the volume of the bone defect (BV) (BS/BV) reflects the structure level of the bone formed (FIGS. 15A and 15B). For control animals, the high ratio (15.10±5.49 mm⁻¹) reflects a rather fine and spread mineralized structure, whereas for positive controls, a lower value represents a more compact bone (5.26±0.36 mm⁻¹). The intermediate values found with the poly(caprolactone-urea-urethane) elastomer matrix alone (7.82±1.65 mm⁻¹) and the bone substitute material according to the invention (composite) (6.99±2.09 mm⁻¹) at 3 months show that the bone produced is being remodelled to find a more compact structure.

### 5.6. Histology

All the data obtained in microtomography is correlated with the local data obtained by histological analysis and carried out successively on the same sample. The femurs removed are fixed in Burckhardt's solution and are included in MMA resin (methyl methacrylate) suitable for hard tissues.

Thick cuts (so that the biomaterial did not tear) were then stained by specific dyes of the different cell types:
Masson (TM) trichrome, stains mineralized tissue in blue, highlights the cells responsible for formation: osteoblasts, and for bone remodelling: osteoclasts, as well as the osteoid mineralization zones (in pink) and blood vessels.
Sudan Black (NS), stains the poly(caprolactone-urea-urethane) elastomer matrix in black.
Sirius Red (RS), stains collagen fibres in red in optical microscopy, and in yellow/orange under polarized light. Allows to visualize the structuring level of a tissue.

### 5.7. Batches Results

### 5.7.1. “Control” Batch

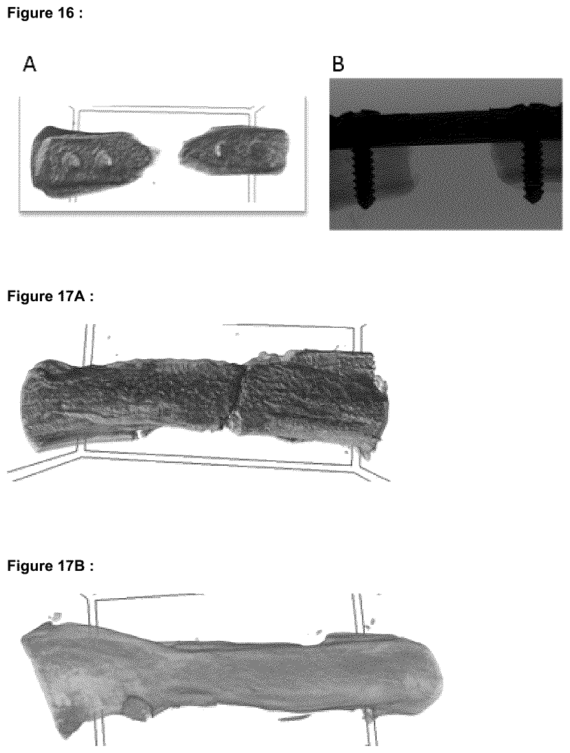

The results obtained are presented in FIG. 16.

The bone defect remains empty, no reconstruction is observed within the defect during the 3 months of the study (FIG. 16). Bone formed at the banks only and a bone synthesis outline is observed for some animals under the fixative (FIG. 16). The defect is filled with fibrous tissue. This confirms the critical size of the bone defect.

### 5.7.2. “Positive Control” (“Contrôle Positif”) Batch

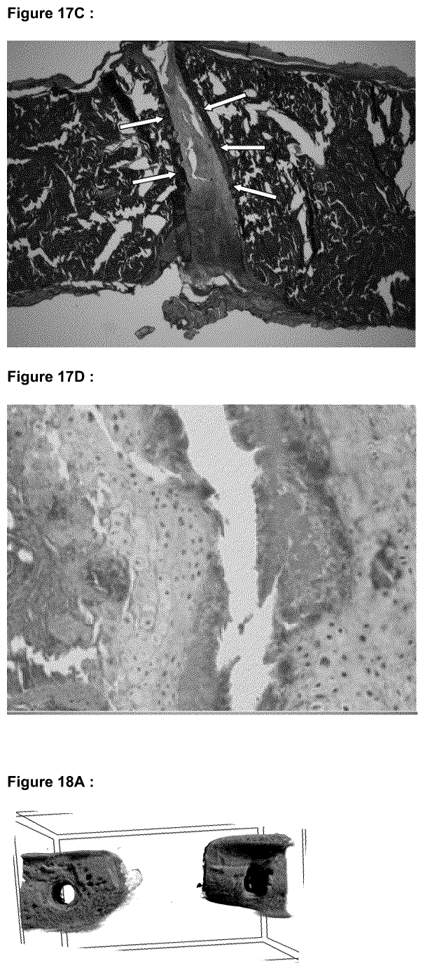

The size of the bone defect is not critical here and is representative of a simple fracture. After 3 months, an almost complete reconstruction is observed when this size is about 2 mm (FIG. 17A). Very active mineralization zones, indicated by the arrows in FIG. 17C, are found again at the bone banks after 1 month, as well as endochondral ossification zones at the center of the defect (FIG. 17D), probably linked to a certain “elasticity” of the system. The rats are active and dynamic, and this zone is subjected to high mechanical stresses.

We can observe in FIG. 17B a complete repair with restoration of bone continuity after 3 months when the size of the defect is less than 1 mm (saw cut only).

The results obtained are presented in FIGS. 17A to 17D.

5.7.3. “Poly(caprolactone-urea-urethane) Elastomer Matrix Alone” Batch

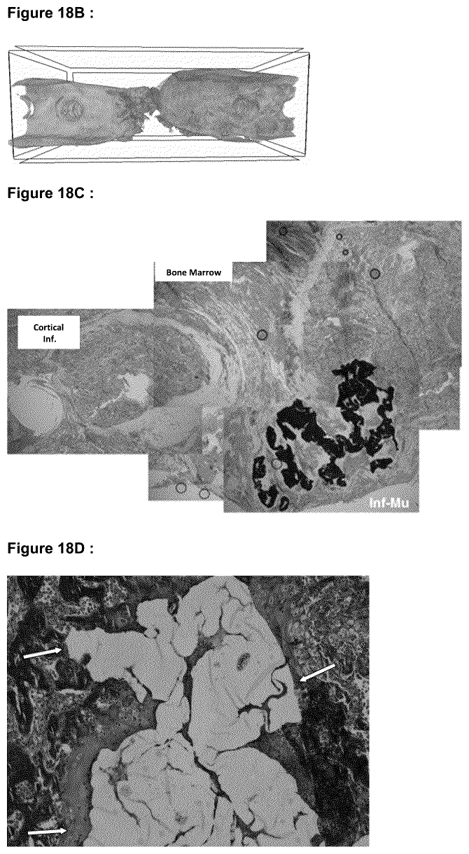

The level of reconstruction is low one month post-implantation (FIG. 18A) and located only at the bone banks. However, stain with Sudan black allows to visualize the poly(caprolactone-urea-urethane) elastomer matrix alone located at the center of the defect and partially integrated into neosynthesised bone (FIG. 18C).

It can be noted that the poly(caprolactone-urea-urethane) elastomer matrix alone has undergone a modification of macroscopic structure: the sponge has “flattened, unrolled” as we observed at one month in a cavity defect, during previous studies. Masson's trichrome staining makes it possible to visualize osteoid areas—corresponding to areas being mineralized by osteoblasts—numerous around this part intimately integrated into the bone (FIG. 18D, indicated by the arrows). In addition, biological material is visible inside the poly(caprolactone-urea-urethane) elastomer matrix alone, which demonstrates a conserved porosity for the latter.

The 3D reconstruction obtained after 3 months (FIG. 18B) reveals a partial reconstruction of the defect, with almost restored continuity. We unambiguously find the osteoinductive nature of the poly(caprolactone-urea-urethane) elastomer matrix alone previously demonstrated on a bone cavity defect model.

However, for certain samples, we have noted a particular trabecular structure of the newformed bone inside the defect in the presence of the poly(caprolactone-urea-urethane) elastomer matrix alone (FIG. 19). In addition, slight bone synthesis is observed at the ends of the fixator, appearing to be “backed” to the latter, located by an arrow in FIG. 19.

### 5.7.4. “Decellularized Bone” Batch

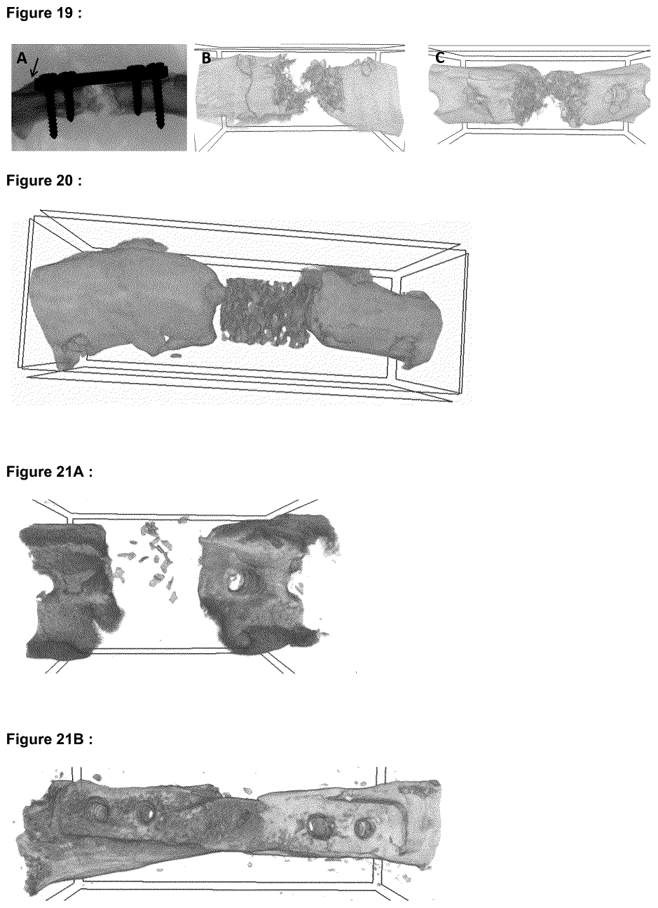

In order to verify the biocompatibility and osteoconduction properties, the bone defect was filled with an Allodyn® R cylindrical rod with a diameter of 2 mm. The implant was cut delicately at the time of its installation so that it completely fills the defect and is in contact with the two bone banks. No reconstruction was observed after 3 months (FIG. 20). A starting resorption is noted for the biomaterial. Moderate bone synthesis around the ends of the fixator (outside the zone to be reconstructed) was observed for a few samples, of a level similar to that observed with the poly(caprolactone-urea-urethane) elastomer matrix alone. No rejection or infection response was recorded.

### 5.7.5. “Bone Substitute Material According to the Invention” Batch

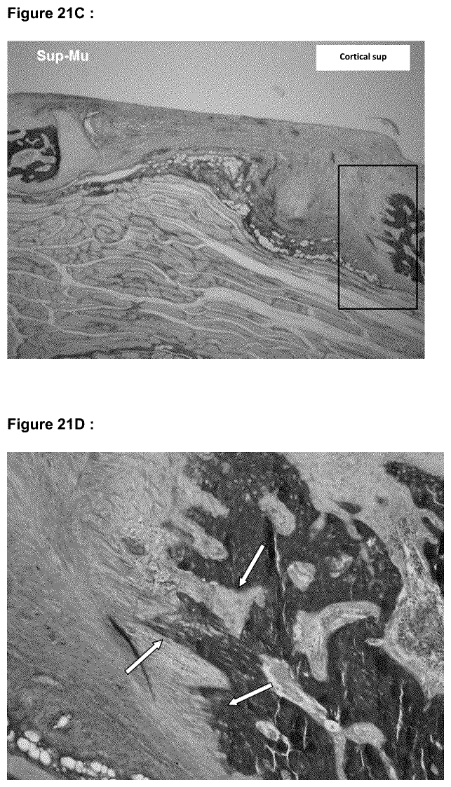

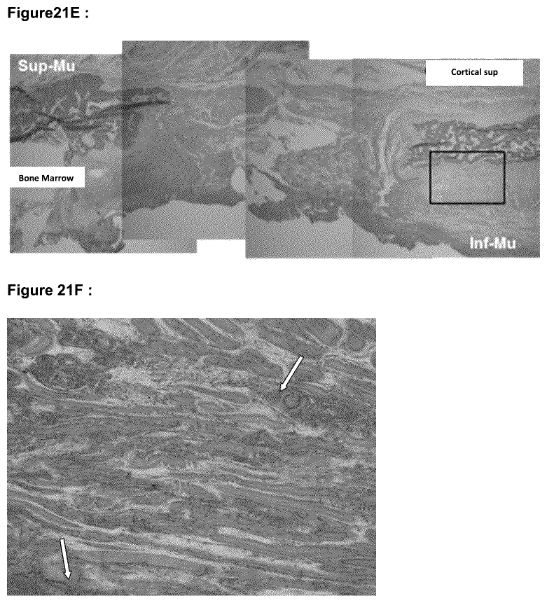

The incorporation of decellularized bone in the form of granules into the poly(caprolactone-urea-urethane) elastomer matrix modifies the reconstruction process. Indeed, after one month, bone is formed on the bone banks, which can be identified by a Masson trichrome staining of bone, with numerous osteoid zones at this level (FIGS. 21C and 21D). However, the area of the bone defect always appears empty; the granules of decellularized bone are clearly visible and are grouped together more or less off-centre with respect to this area (FIG. 21A). However, an intense “delocalized” bone synthesis is noted, with the fixators which become covered with a mineralized layer. This is even more marked after 3 months (FIG. 21B), the fixators being completely covered with bone on their distal and proximal ends. Bone synthesis is intense at this time and the defect area is almost repaired: continuity is restored and the defect almost filled with mineralized tissue in half of the animals. Histological analysis will make it possible to verify the structure of this newly formed bone and its level of remodelling.

For certain samples, mineralized granules are found completely outside the area to be reconstructed, trapped between the bone tissue and the surrounding muscle. Here also, the histological analysis will make it possible to tell whether these are residual granules of decellularized bone which have migrate outside the zone of the defect, without being resorbed, or whether these are external mineralization foci.

Indeed, on certain histological section planes, the presence of more or less diffuse zones during mineralization located outside the bone defect can be seen from one month (FIGS. 21E and 21F).

### CONCLUSIONS

Bone formation generally occurs around and to a smaller scale within the biomaterials. The multi-scale porosity of the porous bone substitute material according to the invention appears to be a positive factor since several ossification foci are detected therein, indicating that differentiated and active cells could migrate in this area, probably accompanied by blood vessels.

These histological analyses also make it possible to visualize the porous bone substitute material according to the invention within the bone defect: this one appears fragmented and partially included in the mineralized tissue, demonstrating degradation and biointegration compatible with repair kinetics. The in vivo lifetimes of the porous bone substitute material according to the invention estimated is 13 to 42 months. This lifetime is compatible with clinical use.

The histological analyses confirm the osteoconductive nature and the degradation of the porous bone substitute material according to the invention, visible from the first month of implantation, as well as the formation of bone. All the results indicate that the porous bone substitute material according to the invention has good mechanical properties, good biocompatibility and degradation adapted to the reconstruction of the bone tissue. It has interesting in vivo performances by inducing intense bone synthesis, until tissue continuity between two bone fragments is restored. The presence of decellularized bone in the porous bone substitute material according to the invention increases the production of mineralized tissue, this one is three times greater than that obtained with the poly(caprolactone-urea-urethane) elastomer matrix alone after three months. However, this intense production is not located only inside the bone defect, and also takes place around the fixators. Several mineralization zones have been detected between the bone tissue and the surrounding muscle.

1. A porous bone substitute material comprising:
at least one elastomer matrix, and
particles of decellularized bone.

2. Porous bone substitute material according to claim 1, characterized in that the at least one elastomer matrix comprises an elastomer based on poly(ester-urea-urethane), the ester being chosen from caprolactone oligomers (PCL), lactic acid oligomers (PLA), glycolic acid oligomers (PGA), hydroxybutyrate oligomers (PHB), hydroxyvalerate oligomers (PVB), dioxanone oligomers (PDO), poly(ethylene adipate) oligomers (PEA), poly(butylene adipate) oligomers (PBA) or combinations thereof.

3. Porous bone substitute material according to claim 1, characterized in that the decellularized bone particles can be obtained from natural bone.

4. Porous bone substitute material according to claim 1, characterized in that the decellularized bone particles have a diameter of between 1 nm and 1 mm.

5. Porous bone substitute material according to claim 1, characterized in that the decellularized bone particles represent at least 10% by weight of the porous bone substitute material.

6. Porous bone substitute material according to claim 1, characterized in that said porous bone substitute material has a multiscale pore size of between 50 μm and 2000 μm.

7. Porous bone substitute material according to claim 1, characterized in that said porous bone substitute material has a total porosity of greater than or equal to 60%.

8. Porous bone substitute material according to claim 1, characterized in that said porous bone substitute material has a volume of between 0.1 and 400 cm³.

9. Porous bone substitute material according to claim 1 for use in bone repair, preferably for repair of a bone cavity defect and/or repair of a segmental bone defect.

10. Porous bone substitute material for use according to claim 9, wherein the bone repair is greater than or equal to 5% by volume of the volume of the bone to be repaired.

11. A bone repair kit comprising the porous bone substitute material according to claim 1 and a fixator.

12. A method for preparing a porous bone substitute material comprising the following steps:
a) preparing an organic phase comprising the compounds required for the synthesis of poly(ester-urea-urethane),
b) adding water and the decellularized bone particles to the organic phase of step a) to form an emulsion,
c) polymerizing/crosslinking the emulsion obtained at step b) to obtain said porous bone substitute material,
d) washing said porous bone substitute material obtained at step c), and
e) drying said porous bone substitute material obtained at step d).
